(19) United States
(12) Patent Application Publication
(10) Pub. No.: US 2026/0325443 A1
(43) Pub. Date: Oct. 22, 2026

(54) OLFACTORY DELIVERY OF THERAPEUTIC PSYCHEDELICS

(71) Applicant: Rocket Science Health Corp., Victoria (CA)

(72) Inventors: Kenneth IRVING, Victoria (CA); Kenza ENRIGHT, Victoria (CA); Sohier HALL, Victoria (CA); Nicholas ALLAN, Victoria (CA); Jacob M. HOOKER, Victoria (CA)

(21) Appl. No.: 19/696,741

(22) Filed: Jun. 3, 2026

Related U.S. Application Data
(63) Continuation of application No. PCT/CA2024/051613, filed on Dec. 3, 2024.
(60) Provisional application No. 63/606,083, filed on Dec. 4, 2023.
(60) Provisional application No. 63/651,644, filed on May 24, 2024.
(60) Provisional application No. 63/715,070, filed on Nov. 1, 2024.

(51) Int. Cl. A61K 9/00; A61K 31/135

(52) U.S. Cl. CPC A61K 9/0043; A61K 31/135

(57) ABSTRACT

The present disclosure describes methods and devices for olfactory delivery of therapeutic psychedelics such as ketamine. Devices and methods of the present disclosure may provide convenient and effective ways to accomplish olfactory delivery of therapeutic psychedelics.

### CROSS REFERENCE

This application is a continuation of International Application No. PCT/CA2024/051613, filed Dec. 3, 2024, which claims the benefit to U.S. Provisional Application No. 63/606,083, filed Dec. 4, 2023, U.S. Provisional Application No. 63/651,644, filed May 24, 2024, U.S. Provisional Application No. 63/715,070, filed Nov. 1, 2024, the contents of which are incorporated herein in their entirety by reference.

### FIELD

The present disclosure relates generally to delivery of psychedelics, and more specifically, delivery via the olfactory.

### BACKGROUND

Certain psychedelics are known to provide therapeutic effects to subjects. Ketamine, and similar psychedelic therapeutics have been found to treat a wide variety of disorders related to the central nervous system. While therapeutic effects of psychedelics on the central nervous system (or a region, tissue, or organ thereof) are of interest, delivery is known to present challenges. Olfactory delivery is a possibility for therapeutic psychedelics.

The therapeutic potential of drugs targeting the brain through olfactory neural pathways via intranasal application is significant, including decreased systemic absorption, smaller doses, and reduced risk of complications from over-medication. Nasal mist devices are a standard method to deliver drugs to the nasal cavity. Recent research has established the inefficiency of these methods compared to methods designed to deliver drugs more focally into the nasal cavity where drug action is desired. Focal delivery promises to deposit the medication bolus directly to the olfactory cleft immediately below the cribriform plate where the olfactory nerves pass into the brain, whereas standard nasal misters deposit drug dosage throughout the nasal cavity and sinuses with potential significant dilution prior to reaching the olfactory cleft, due to dispersion over a large surface area. Moreover, spray devices typically atomize or vaporize the drug compound, creating small droplets that are degraded by high velocity airflow through the nasal valve. Shear forces can destroy some drug molecules and biologics as they are atomized into droplets. With few exceptions, existing devices typically deliver only 5-8% of a dose to the olfactory cleft. There remains a need for formulations, methods, and devices for targeted delivery of therapeutic psychedelics to subregions of the nasal cavity, such as the olfactory cleft.

### SUMMARY

It is appreciated by the inventors that challenges related to delivery of therapeutic psychedelics to certain regions of the central nervous system include difficulty in crossing the blood-brain barrier. For example, the blood brain barrier and/or the blood-cerebrospinal fluid barrier may limit or prevent psychedelics from having therapeutic effects when they are delivered intravenously. Intranasal delivery efficiencies have typically been low, even for non-protein, small-molecule therapeutics. Challenges associated with olfactory delivery of psychedelics include difficulties in accommodating for variations in the nasal channel, difficulties in targeting deposition of the psychedelic at the correct location in the nasal channel, and difficulties providing a device which can reliably and repeatably deliver a composition to locations within the nasal channel with a high degree of precision across a population of subjects. Methods and devices of the present invention provide for targeted delivery of therapeutic psychedelics to the olfactory region such that the therapeutic psychedelics are not hindered by off-target delivery, thereby improving delivery efficiency and therapeutic efficacy. Additional advantages of the present invention may include the avoidance of needles (particularly relevant to subjects with needle-phobia), the possibility of self-administration, and enablement of the delivery of formulations with viscosity too high for intravenous injection.

It is appreciated by the inventors that difficulties in nasal delivery include anatomical, cognitive, and dexterity related challenges related to providing a reliable and effective nasal delivery device. For example, the internal nasal valve is a flow-limiting segment of the nasal channel bounded medially by the dorsal septum, laterally by the caudal portion of the upper lateral cartilage and inferiorly by the head of the inferior turbinate that together present a physical barrier between the nasal vestibule and the rest of the nasal channel, including the respiratory region and the olfactory cleft. The internal nasal valve is bounded by nasal tissue that can swell or block the path to the nasal channel, which varies based on time of day, environmental factors, and genetically among individuals. Devices may need to be inserted at particular angles or depths in order to target a specific area, and users may struggle with correct placement and actuation of a device. Users with motor skill impairment (e.g., persons with Parkinson's disease, arthritis) or cognitive impairments (e.g., Alzheimer's disease) may especially struggle to correctly articulate, position, and actuate an intranasal delivery device.

To counter such difficulties, this disclosure provides formulations, methods, and devices for olfactory delivery of therapeutic psychedelics such as ketamine. This disclosure provides an introducer device for targeted delivery of a psychedelic-containing composition to a target region of a nasal cavity of a subject, which can readily be actuated and positioned by users of various patient populations, and reliably deliver the composition to a target sub-region of the nasal cavity, to enable precision delivery of the psychedelics. As a non-limiting example, the olfactory cleft can be a target sub-region of the nasal cavity, such that non-spray, targeted delivery of a therapeutic to the olfactory cleft produces a desired effect. Additional target sub-regions of the nasal cavity may be identified.

In one aspect is a method of olfactory delivery of therapeutic psychedelics, the method comprising: selectively delivering a formulation comprising a therapeutically effective amount of the therapeutic psychedelic to an olfactory region of the subject; wherein at least a portion of a formulation is delivered to one or more of the group consisting of cerebrospinal fluid, the central nervous system or a region, tissue, or organ thereof. In some embodiments, the therapeutic psychedelic may be delivered to a target or organ system other than the CNS. Non-limiting examples include the lymphatic system, Nasal Associated Lymphatic Tissue (NALT), and Cribriform Lymphatic/Glymphatic, Systemic, etc. Therapeutic psychedelics may also elicit an immune response in the CNS.

In some embodiments, the formulation is ejected from a dispensing element, wherein at least about 70%, at least about 75%, at least about 80%, at least about 85%, at least about 90%, or at least about 95% of the formulation passes through a circular opening having a diameter of between about 5 mm and about 6 mm at a distance of about 25 mm from where the formulation is ejected from the dispensing element. In some embodiments, the formulation has a viscosity of between about 0.5 cP and about 100 cP. In some embodiments, the formulation has a viscosity of between about 0.5 cP and about 50 cP. In some embodiments, the formulation has a viscosity of between about 0.5 cP and about 10 cP. In some embodiments, the formulation has a viscosity of between about 45 cP and about 55 cP. In some embodiments, the formulation is ejected from the dispensing element at a velocity of between about 1 m/s and about 30 m/s. In some embodiments, the formulation is ejected from the dispensing element at a velocity of between about 2 m/s and about 4 m/s. In some embodiments, the formulation is ejected from the dispensing element at a velocity of between about 25 m/s and about 30 m/s. In some embodiments, the formulation is ejected from a dispensing element, wherein at least 75% of the formulation passes through a circular opening having a diameter of between about 5 mm and about 6 mm at a distance of about 25 mm from where the formulation is ejected from the dispensing element, wherein the formulation has a viscosity of between about 0.5 cP and about 50 cP, and wherein the formulation is ejected from the dispensing element at a velocity of between about 1 m/s and about 30 m/s.

In some embodiments, at least about 50%, at least about 60%, at least about 70%, at least about 80%, or at least about 90% of the formulation is delivered to the olfactory region of the subject. In some embodiments, delivery of the therapeutic psychedelic to the olfactory region of the subject results in a plasma concentration of the therapeutic psychedelic about 30 min after delivery that is at least about 2 times, at least about 4 times, at least about 6 times, at least about 8 times, or at least about 10 times a plasma concentration of the therapeutic psychedelic when about the same dose is delivered intravenously.

In some embodiments, the formulation is delivered as a laminar flow. In some embodiments, the formulation is delivered as a liquid jet. In some embodiments, the formulation is delivered as a flow having a Reynold's number of 2300 or less. In some embodiments, the delivering of the formulation as a laminar flow, a liquid jet, or a flow having a Reynold's number of 2300 or less: a) increases on target delivery of the composition to the target region, b) decreases off target delivery of the composition to the nasal cavity, or c) both, compared to delivering the formulation with a spray ejection profile.

In some embodiments, selectively delivering the formulation to the olfactory region of the subject comprises ejecting the formulation from an ejection zone in a nasal cavity of the subject, wherein the ejection zone is: (a) 0 mm to 30 mm superior to a horizontal line that intersects the anterior aspect of the internal nasal valve, and (b) 0 mm to 20 mm anterior to an inclined line that intersects the anterior aspect of the middle turbinate and the posterior aspect of the vestibule. In some embodiments, the ejection zone is further: i. 0 mm to 40 mm inferior to a horizontal line that is parallel to the inferior aspect of the olfactory cleft, ii. 0 mm to 20 mm posterior to the internal nasal dorsum, iii. 10 mm to 50 mm superior to a horizontal line that intersects the inferior aspect of the columella, iv. 0 mm to 30 mm superior to a horizontal line that intersects the superior aspect of the inferior turbinate, v. 0 mm to 3 mm from the septum, or vi. any combination thereof. In some embodiments, delivering the formulation from the ejection zone: a) increases on target delivery of the composition to the target region, b) decreases off target delivery of the composition to the nasal cavity, or c) both, compared to dispensing the formulation outside the ejection zone.

In some embodiments, the formulation is delivered by a device comprising the dispensing element, wherein the device is inserted into a nasal cavity of the subject at an angle of between about 30 degrees and about 40 degrees from a vertical line, wherein the vertical line is perpendicular to a horizontal line that is parallel to an inferior aspect of the olfactory cleft of the subject.

In some embodiments, the formulation delivered to the olfactory region remains at the olfactory region for at least about 10 seconds, at least about 30 seconds, at least about 1 min, at least about 2 min, at least about 3 min, at least about 4 min, at least about 5 min, at least about 6 min, at least about 7 min, at least about 8 min, at least about 9 min, at least about 10 min, at least about 11 min, at least about 12 min, at least about 13 min, at least about 14 min, or at least about 15 min.

In some embodiments, the therapeutic psychedelic is ketamine, R-ketamine, S-ketamine, psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, or a serotonin antagonist. In some embodiments, the therapeutic psychedelic is an analog of one of the group consisting of: ketamine, R-ketamine, S-ketamine, psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, and a serotonin antagonist.

In some embodiments, the therapeutic psychedelic is ketamine, or a derivative or analog thereof.

In some embodiments, the therapeutic psychedelic is conjugated to a therapeutic agent. In some embodiments, the therapeutic agent is a drug or a radioisotope.

In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the therapeutic psychedelic in the formulation is delivered to one or more of the group consisting of cerebrospinal fluid, the central nervous system, and a region, tissue, or organ thereof.

In some embodiments, the method provides delivery of a greater amount of the therapeutic psychedelic to one or more of the group consisting of cerebrospinal fluid, the central nervous system, and a region, tissue, or organ thereof compared to intravenous delivery of an identical dose of the therapeutic psychedelic. In some embodiments, the therapeutic psychedelic provides a therapeutic effect upon delivery to the cerebrospinal fluid, the central nervous system or region, tissue, or organ thereof. In some embodiments, the subject has been diagnosed with a disease or disorder related to the central nervous system. In some embodiments, delivery of the therapeutic psychedelic provides effective treatment for Alzheimer's disease, Parkinson's disease, a cancer of the brain, a cancer of the CNS, a non-CNS disorder, an immunological disorder, an oncological disorder, a hematological disorder, an opthalmological disorder, a neurological disorder, a respiratory disorder, a cardiovascular disorder, an infectious disease, vitamin deficiency, multiple sclerosis, improvement of uterine contractions, postmenopausal osteoporosis, cryptorchism, endometriosis, central precocious puberty (CPP), Primary nocturnal enuresis, central cranial diabetes insipidus, nocturia, cranial diabetes insipidus or nocturia associated with multiple sclerosis, stroke, epilepsy, central nervous system trauma, a metabolic disorder, an infectious agent (i.e. Naegleria fowleri) or protein (i.e. Creutzfeldt Jacobs disease), a malignant tumor, a benign tumor, glioblastoma, anosmia, or an endocrine disorder. In some embodiments, delivery of the therapeutic psychedelic provides effective treatment for depression, treatment-resistant depression (TRD), post-traumatic stress disorder (PTSD), alcohol use disorder (AUD), generalized anxiety disorder (GAD), bipolar disorder, bipolar disorder II (BDII), postpartum depression (PPD), fragile X syndrome, opioid withdrawal, anorexia nervosa, bulimia nervosa, binge eating disorder, major depressive disorder (MDD), fibromyalgia, acute suicidal ideation and behavior (ASIB), cluster headaches, obsessive compulsive disorder, adjustment disorder, attention deficit hyperactivity disorder (ADHD), ischemic stroke, or autism spectrum disorder (ASD).

In some embodiments, the formulation is delivered without exposing the therapeutic psychedelic to shear forces sufficient to damage a significant portion of the therapeutic psychedelic, without aerosolizing the formulation, or by passing the formulation through a shear disintegrating tip. In some embodiments, the therapeutic psychedelic bypasses the blood-brain barrier, the blood-cerebrospinal fluid barrier, or the arachnoid membrane. In some embodiments, the non-CNS-target is blood.

In some embodiments, the formulation is delivered via a device comprising: a. a housing defining first and second insertable portions, each for insertion into a nasal channel of the subject, wherein, upon insertion of at least one of the insertable portions into the nasal channel of the subject, the at least one insertable portion engages tissue within the nasal channel to open or expand an internal nasal valve of the subject thereby positioning the at least one insertable portion for delivery of the formulation to the olfactory region of the subject; and b. an actuator which delivers the formulation from at least one of the insertable portions when the device is actuated.

In another aspect is a device for olfactory delivery of therapeutic psychedelics, the device comprising: a housing comprising an insertable portion comprising a distal end, and a proximal end; and a subject-engaging portion which engages a columella region of the subject to seat the di stal end of the insertable portion within an ejection zone of a nasal channel of the subject; wherein the device is configured to deliver a formulation comprising a therapeutically effective amount of the psychedelic to the olfactory region of the subject.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description and accompanying drawings, that set forth illustrative embodiments in which the principles of the present disclosure are utilized.

FIG. 1A depicts a bottom view of an exemplary embodiment of a subject's nose.

FIG. 1B depicts a side view of an exemplary embodiment of a subject's nose.

FIG. 1C depicts a side view of a representative subject's nasal channel.

FIG. 1D depicts a side view of an exemplary embodiment of a representative subject's nasal channels from the vestibule to the olfactory cleft based on a posteriorly oriented plane.

FIG. 1E depicts a side view of an exemplary embodiment of a representative subject's nasal channels from the vestibule to the olfactory cleft based on an anteriorly oriented plane.

FIG. 1F depicts a side view of an exemplary embodiment of a representative target ejection zone, according to some embodiments.

FIG. 1G depicts a front, base and perspective view of an exemplary embodiment of a subject's nose and the columella region.

FIG. 1H depicts a side view of an exemplary embodiment of a representative subject's nasal cavity, including internal anatomical features.

FIG. 1I depicts a side view of an exemplary embodiment of a representative subject's sinus, according to some embodiments.

FIG. 1J depicts a side view of a subject's nasal cavity.

FIG. 2A depicts an exemplary embodiment of an Exemplary Device in a second configuration, according to some embodiments.

FIG. 2B depicts an exemplary embodiment top view of an Exemplary Device in the second configuration, according to some embodiments.

FIG. 2C depicts an exemplary embodiment trigger button side view of an Exemplary Device in the second configuration, according to some embodiments.

FIG. 2D depicts an exemplary embodiment chassis side view of an Exemplary Device in the second configuration, according to some embodiments.

FIG. 2E depicts an exemplary embodiment bottom view of an Exemplary Device in the second configuration, according to some embodiments.

FIG. 2F depicts an exemplary embodiment perspective view of an Exemplary Device in the first configuration, according to some embodiments.

FIG. 2G depicts an exemplary embodiment front view of another Exemplary Device in the first configuration, according to some embodiments.

FIG. 2H depicts an exemplary embodiment front view of an Exemplary Device in the second configuration, according to some embodiment.

FIG. 2I depicts an exemplary embodiment front view of another Exemplary Device in the first configuration, according to some embodiments.

FIG. 2J depicts an exemplary embodiment front view of an Exemplary Device in the second configuration, according to some embodiment.

FIG. 3A depicts an exemplary embodiment of an Exemplary Device in the second configuration with one dispensing element revealed and a side view of an exemplary embodiment of a subject's nose, according to some embodiments.

FIG. 3B depicts an exemplary embodiment of an Exemplary Device in the second configuration with one dispensing element revealing along the passageway and a side view of an exemplary embodiment of a representative subject's nasal channels from the vestibule to the olfactory cleft based on an anteriorly oriented plane, according to some embodiments.

FIG. 4A depicts an exemplary embodiment of a cartesian reference plane of (left to right) a front, a top, and a side view of an Exemplary Device., according to some embodiments.

FIG. 4B depicts an exemplary embodiment of the subject plane, according to some embodiments.

FIG. 4C depicts an exemplary embodiment of (left to right) a front Coronal Plane, a top Transverse Plane, and a back Sagittal Plane view, according to some embodiments.

FIG. 4D depicts an exemplary embodiment of an Exemplary Device Sagittal Angle positioning in the subject, according to some embodiments.

FIG. 4E depicts an exemplary embodiment of an Exemplary Device Coronal-Medial Angle positioning in the subject, along a front view of the Coronal Plane, and a top view along a transverse plane, according to some embodiments.

FIG. 4F depicts an exemplary embodiment of an Exemplary Device Depth positioning along a front view of the Coronal Plane, and along a side view of the Sagittal Plane in the subject, according to some embodiments.

FIG. 4G depicts an exemplary embodiment of an Exemplary Device along a side view of the sagittal plane of delivery to the target region in the subject, according to some embodiments.

FIG. 4H depicts an exemplary embodiment of an Exemplary Device along a side view of the sagittal plane aiming from the respiratory region in the subject, according to some embodiments.

FIG. 4I depicts a side view of a target ejection zone, according to some embodiments.

FIG. 4J depicts a side view of a target ejection zone with respect to other nasal cavity anatomy, according to some embodiments.

FIGS. 5A-C depict images of a transparent model nasal cavity with a colored solution demonstrating delivery to an olfactory region of the model.

FIG. 6A depicts a mouse in an olfactory delivery study with a catheter inserted into the nostril to deliver a formulation to the olfactory region.

FIG. 6B depicts a diagram of the targeted olfactory delivery shown in FIG. 6A.

FIG. 7A depicts a mouse in an intranasal delivery study with a pipet tip inserted into the nostril to deliver a formulation to the nasal cavity.

FIG. 7B depicts a diagram of the non-targeted intranasal delivery shown in FIG. 7A.

FIG. 8 shows images of flow time series for olfactory delivery of technetium-99.

FIG. 9 depicts comparative concentrations of ketamine sampled from plasma, brain tissue, and the olfactory bulb 15 minutes after administration of ketamine (5 mg/kg) to rats via olfactory delivery (OD), intranasal (IN) delivery, and intravenous (IV) delivery.

FIG. 10A depicts a time course of ketamine-HCl plasma concentration following intravenous (IV) or olfactory delivery (OD) administration.

FIG. 10B depicts comparison of ketamine-HCl concentration in the brain, olfactory bulb, and plasma at t=200 min. following intravenous (IV) or olfactory delivery (OD) administration.

### DETAILED DESCRIPTION

This disclosure provides methods and devices for olfactory delivery of therapeutic psychedelics. As used herein, the term “psychedelic” refers to a substance which is psychoactive or produces a change in perception, mood, or cognitive process when administered at a sufficient dose. Non-limiting examples of psychedelics include ketamine, R-ketamine (arketamine), S-ketamine (esketamine), psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, a serotonin antagonist, and analogs or derivatives thereof.

The psychedelic can be of natural or synthetic origin.

This disclosure provides an introducer device for targeted delivery of a composition to a target region of a nasal cavity of a subject, which can be readily actuated and positioned by users of various patient populations, and reliably deliver the composition to the target region of the nasal cavity. Exemplary devices utilize a dual nostril inserter with a columella engaging portion positioned therebetween in order to quickly and reliably seat the insertable portions of the device within a target ejection zone of a subject's nasal cavity. The exemplary devices disclosed herein can permit for quick, easy, and reliable positioning of a dispensing element within the nasal channel so as to permit for accurate and targeted deposition of compositions to target regions of the nasal cavity. Further, exemplary devices disclosed herein can enable quick, easy, and reliable positioning and targeted deposition across diverse patient populations (e.g., users that are elderly, cognitively impaired, dexterity impaired, or have variations in nasal anatomy), by users of varying skill (e.g., untrained bystanders), and under high stress circumstances (e.g., a medical emergency), which may otherwise prevent proper use of intranasal delivery devices.

The devices of the present disclosure may comprise housing comprising one or a combination of the following: a subject engaging portion, one or more insertable portions, one or more dispensing elements, and a trigger.

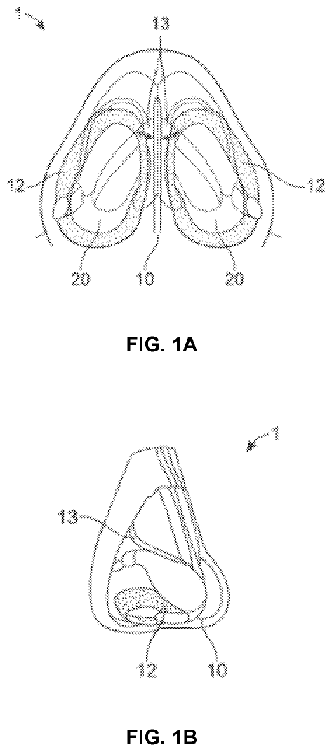

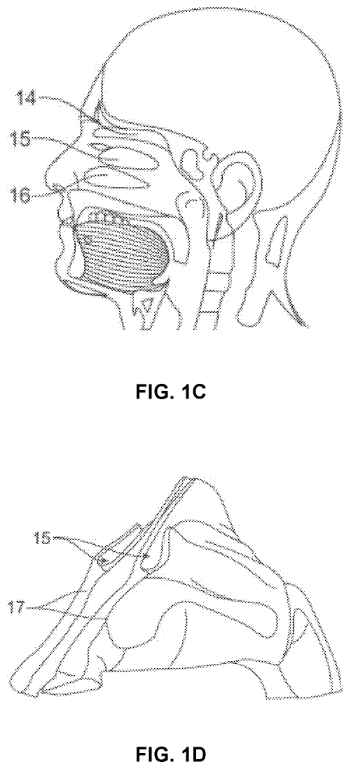

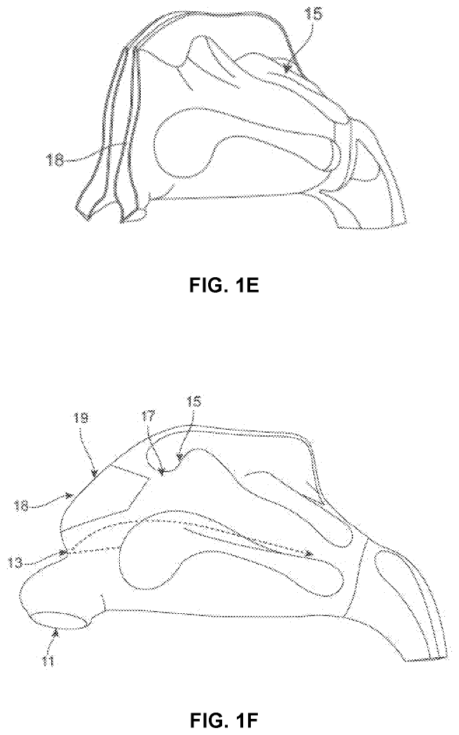

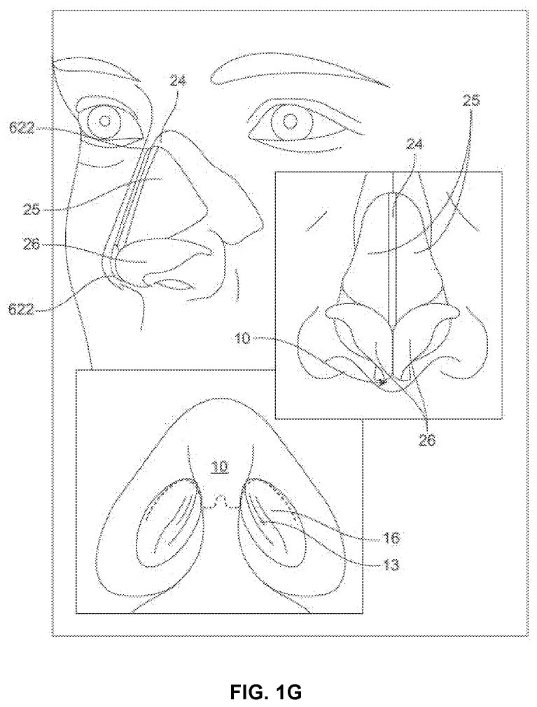

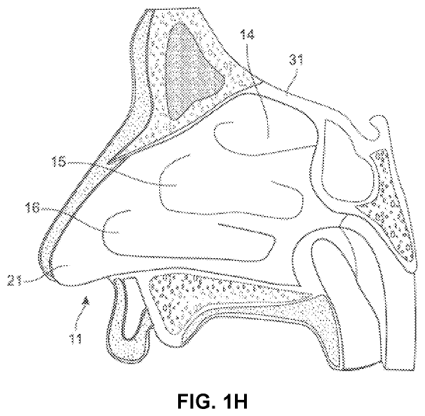

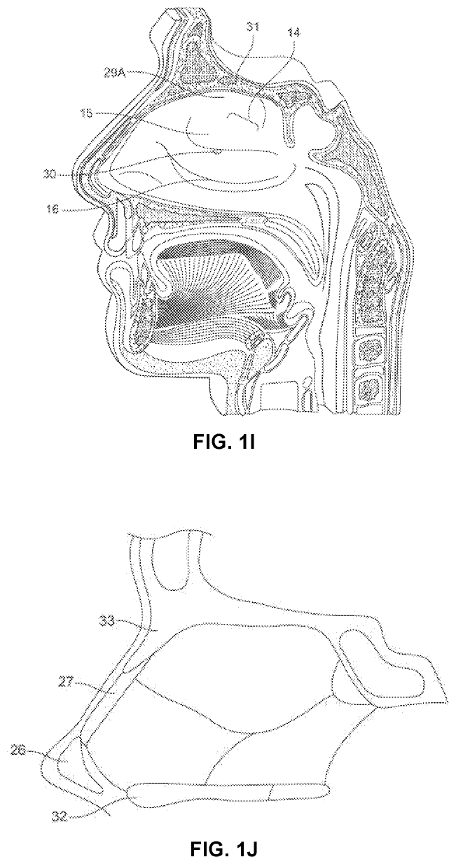

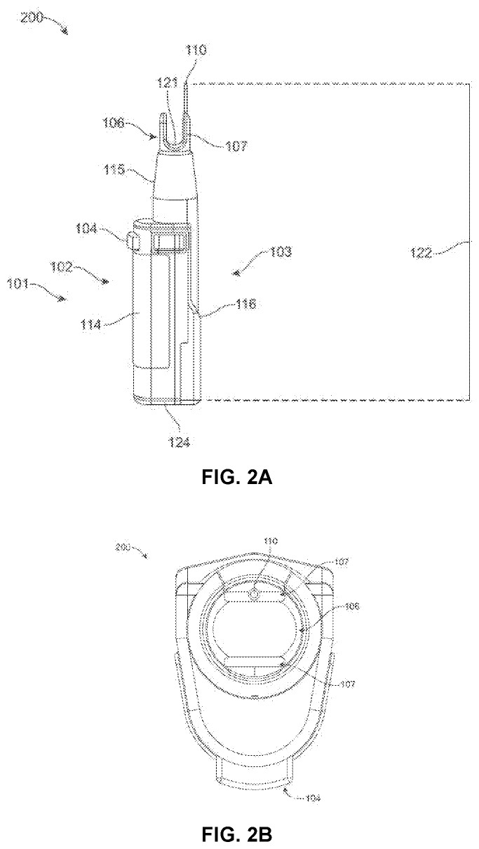

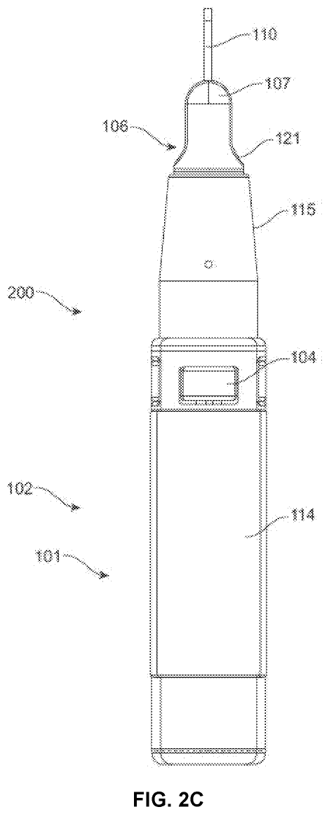

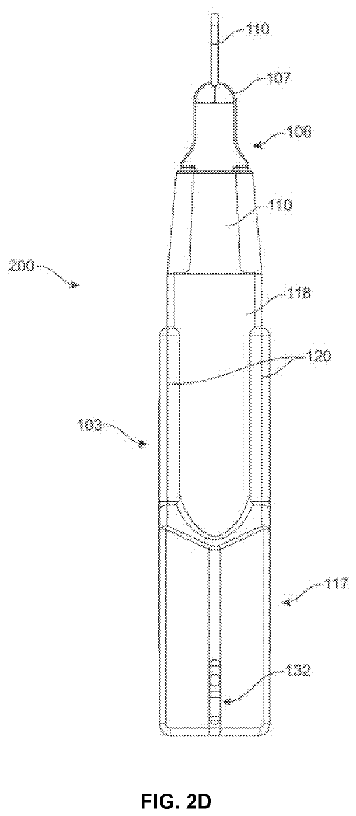

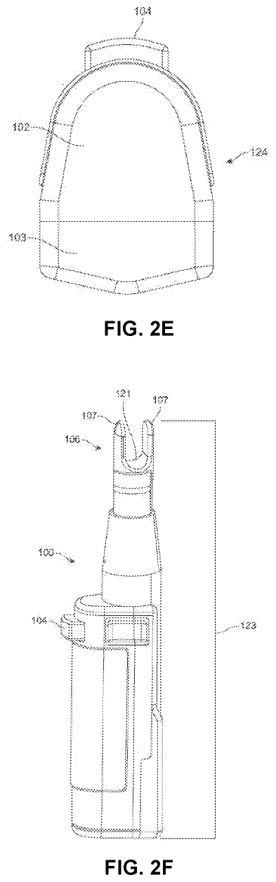

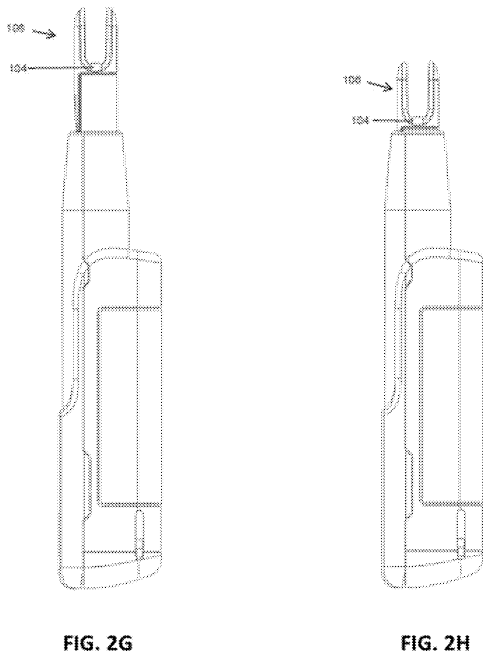

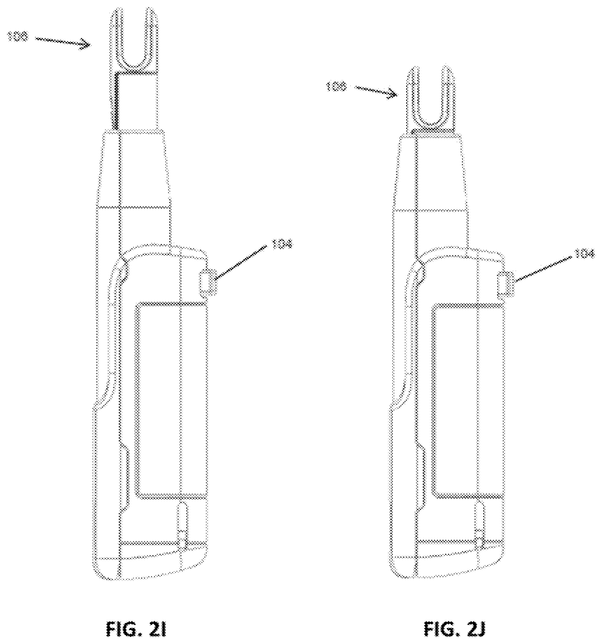

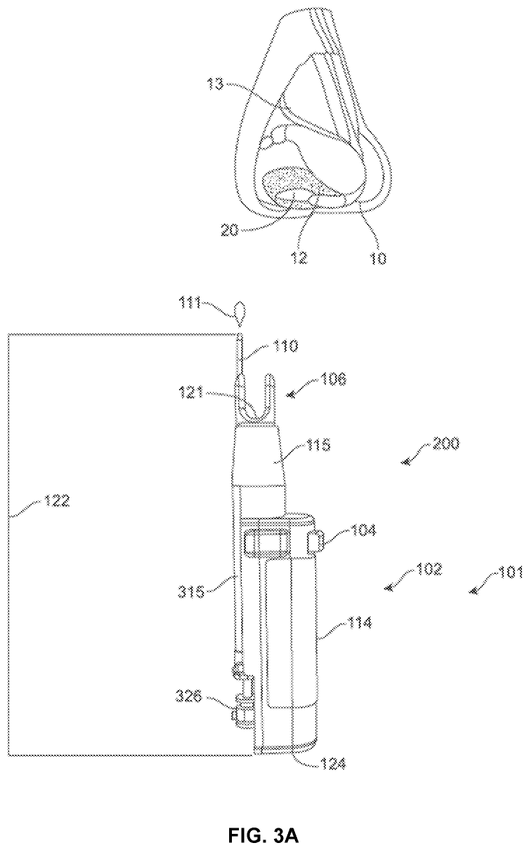

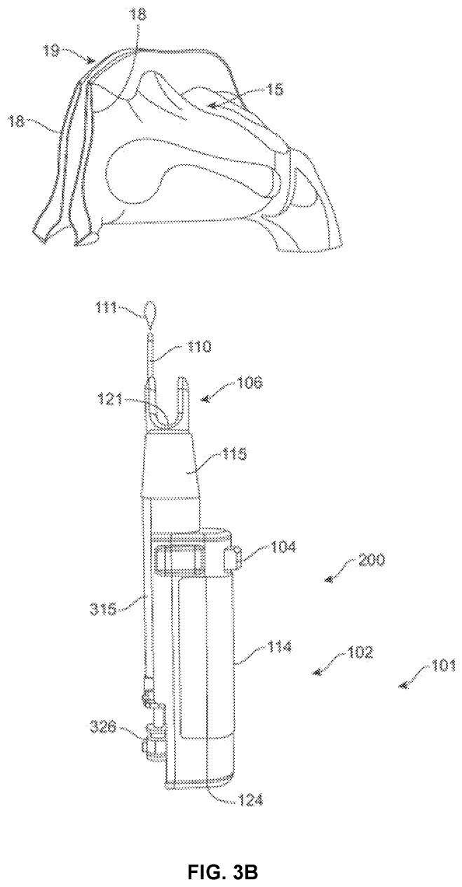

FIG. 1A depicts a bottom view of an exemplary embodiment of a subject's nose. FIG. 1B depicts a side view of an exemplary embodiment of a subject's nose. In some embodiment's the nose 1 has a columella region 10 between the entrance to two nasal channels 20. In some embodiments, the nose 1 has an external nasal valve 12 coupled to the nasal channel 20. In some embodiments, the nose 1 has an internal nasal valve 13 (INV) coupled to the nasal channel 20.

FIG. 1C depicts a side view of an exemplary embodiment of a subject's nasal cavity showing an inferior turbinate 16, a middle turbinate 15, and a superior turbinate 14. FIG. 1D depicts an angled side view of an exemplary embodiment of a representation subject's nasal channels 20 from the vestibules 21 to the olfactory clefts 23 based on a posteriorly oriented plane 17 showing the middle turbinates 15. FIG. 1E depicts an angled side view of an exemplary embodiment of a representation subject's nasal channels 20 from the vestibules to the olfactory clefts based on an anteriorly oriented plane 18. FIG. 1F depicts a side view of an exemplary embodiment of a representation subject's target ejection area 19. FIG. 1G depicts a front, base and perspective view of an exemplary embodiment of a representation subject's nose exposing the columella region 10. The respiratory regions comprise turbinates that present physical obstacles to delivery to the upper reaches of a nasal channels 20, e.g., the olfactory clefts 23. Each respiratory region comprises at least one superior turbinate 14. Each respiratory region comprises at least one middle turbinate 15. Each respiratory region comprises at least one inferior turbinate 16. Each respiratory region comprises at least one posterior pathway 17 that involves at least one middle turbinate 15. Each respiratory region comprises at least one anterior pathway 18 that does not involve at least one middle turbinate 15.

In some cases, the middle turbinate 15 comprises a physical obstruction for composition delivery to an olfactory cleft. In some cases, the middle turbinate 15 comprises a most anterior aspect about aligned with the cheek bone. In some embodiments, the middle turbinate 15 comprises a most anterior aspect not aligned with the cheek bone. In some cases, the nasal channels 20 simplify anteriorly, and comprise angled pathways without one or more turbinates presenting physical obstacles to delivering a composition 111 to the upper nasal channels, including the olfactory clefts 23, or directing compositions 111 down one or more meatuses, e.g., the middle meatus 30, to the nasopharynx. In some cases, the nasal channel 20 comprises one pathway from the vestibule 21 to the olfactory cleft 23 based on an anteriorly oriented plane 18 with a target ejection point 19. In some embodiments, the nose comprises the nasal septum 24, upper lateral cartilage 25, and lower lateral cartilage 26.

FIG. 1H depicts a side view of an exemplary embodiment of a representation subject's nasal cavity. In some cases, the subject's nasal cavity 11 comprises the nasal vestibule 21, inferior turbinate 16, middle turbinate 15, superior turbinate 14, cribriform plate 31 or a combination thereof.

FIG. 1I depicts a side view of an exemplary embodiment of a representation subject's sinus. In some cases, the subject's sinus comprises the inferior turbinate 16, middle turbinate 15, superior turbinate 14, cribriform plate 31, middle meatus 30, or a combination thereof.

FIG. 1J depicts a side view of an exemplary embodiment of a representation subject's nasal cavity. In some embodiments, the nasal cavity 11 comprises the nasal bone 33, septal-lateral cartilage junction 27, lower lateral cartilage 26, anterior nasal spine 32, or a combination thereof.

The “columella” is the firm tissue bridge that separates the nostrils at the base of the nose. The “columella” is the most anteroinferior portion of the nasal septum. The term “columella” or “columella region” is the subnasale, or an anterior nasale spine, or a combination thereof. The columella region may comprise a subnasale, or a combination thereof. The columella shape may be defined by an anterior nasal spine located posteriorly to the columella.

The “introduction pathway” is, in sequence, the vestibule, the anterior aspect of the internal nasal valve, and the anterior aspect of the respiratory region-anterior of the turbinates.

The “internal nasal valve” (INV) is a space bounded medially by the dorsal septum 24 (or just septum), laterally by the caudal portion of the upper lateral cartilage, and inferiorly by the head of the inferior turbinate.

The term “nasal cavity” includes two nasal channels, each comprises a vestibule, respiratory region and olfactory cleft, and a nasopharynx.

The term “turbinates” refers to superior turbinate, middle turbinate, or inferior turbinate, or a combination thereof.

Nasal Cavity: This is the large, air-filled space behind the nose, where air passes on its way to the throat during inhalation.

Internal Nasal Valve: This is the narrowest part of the nasal airway, located just beyond the nostril. It's formed by the edge of the nasal septum, the upper lateral cartilage, and the floor of the nose. The internal nasal valve plays a critical role in regulating airflow through the nose. The area of interest is superior (above) to this structure.

Nasal Septum: This is the thin wall of bone and cartilage that separates the right and left nostrils. It forms the medial (towards the middle) boundary of the region of interest.

Lateral Nasal Wall: This is the side wall of the nasal cavity, which is opposite to the nasal septum. It's a complex structure that includes the turbinates (long, curled bones that protrude into the nasal cavity) and the meatuses (grooves or channels between the turbinates). The lateral nasal wall forms the lateral (towards the side) boundary of the region of interest.

Middle and Superior Meatuses: These are the spaces within the nasal cavity located between the turbinates. The middle meatus is located beneath the middle turbinate and above the inferior turbinate, and the superior meatus is located beneath the superior turbinate. The region of interest encompasses parts of these spaces.

Nostrils (External Nares): These are the two openings of the nose where air enters.

Nasal Vestibule: The nasal vestibule is the most anterior part of the nasal cavity, just inside the nostrils. It's the area of the nose that protrudes outside the face predominantly. This area is lined with skin and contains hair follicles, and it acts as the initial filtering and warming area for inhaled air before it moves deeper into the nasal cavity. The nasal vestibule extends posteriorly to the nasal valve, which is the narrowest part of the nasal airway and located just beyond the nostril.

Nasal Septum: This is a thin wall made of bone and cartilage that separates the left and right sides of the nasal cavity.

Turbinates (Nasal Conchae): These are three pairs of bony projections (inferior, middle, and superior) covered in mucous membrane that protrude into the nasal cavity from the lateral walls. They increase the surface area of the nasal cavity, aiding in the warming, humidification, and filtration of inhaled air.

Meatuses: These are the spaces located between the turbinates. Each turbinate has a corresponding meatus underneath it (i.e., inferior, middle, and superior meatus).

Olfactory Region: This is a small area located at the top of the nasal cavity, where the sense of smell is located.

### I. METHOD

In one aspect, provided herein is a method of delivering a therapeutic psychedelic to a subject in need thereof, the method comprising: a. selectively delivering a formulation comprising a therapeutically effective amount of the therapeutic psychedelic to an olfactory region of the subject; wherein at least a portion of formulation is delivered to one or more of the group consisting of cerebrospinal fluid, the central nervous system or a region, tissue, or organ thereof, and a non-CNS target. In one aspect, provided herein is a method of delivering a therapeutic psychedelic to a subject in need thereof. The method may comprise delivering a formulation comprising a therapeutically effective amount of the therapeutic psychedelic to an olfactory region of the subject. At least a portion of the formulation may be delivered to the cerebrospinal fluid. At least a portion of the formulation may be delivered to the central nervous system. At least a portion of the formulation may be delivered to a region, tissue, or organ of the central nervous system. At least a portion of the formulation may be delivered to a non-CNS target. At least a portion of the formulation may be delivered to one or more of the group consisting of cerebrospinal fluid, the central nervous system, or a region, tissue, or organ thereof, and a non-CNS target.

The method can deliver at least 50% of the formulation to the olfactory region.

In some embodiments, at least about 50% of the formulation is delivered to the olfactory region of the subject. In some embodiments, at least about 55% of the formulation is delivered to the olfactory region of the subject. In some embodiments, at least about 60% of the formulation is delivered to the olfactory region of the subject. In some embodiments, at least about 65% of the formulation is delivered to the olfactory region of the subject. In some embodiments, at least about 70% of the formulation is delivered to the olfactory region of the subject. In some embodiments, at least about 75% of the formulation is delivered to the olfactory region of the subject. In some embodiments, at least about 80% of the formulation is delivered to the olfactory region of the subject. In some embodiments, at least about 85% of the formulation is delivered to the olfactory region of the subject. In some embodiments, at least about 90% of the formulation is delivered to the olfactory region of the subject. In some embodiments, at least about 95% of the formulation is delivered to the olfactory region of the subject.

The formulation can be ejected from a dispensing element. In some embodiments, at least about 70% of the formulation can pass through a circular opening having a diameter of between about 5 mm and 6 mm placed about 25 mm from where the formulation is ejected from the dispensing element. In some embodiments, at least about 75% of the formulation can pass through a circular opening having a diameter of between about 5 mm and 6 mm placed about 25 mm from where the formulation is ejected from the dispensing element. In some embodiments, at least about 80% of the formulation can pass through a circular opening having a diameter of between about 5 mm and 6 mm placed about 25 mm from where the formulation is ejected from the dispensing element. In some embodiments, at least about 85% of the formulation can pass through a circular opening having a diameter of between about 5 mm and 6 mm placed about 25 mm from where the formulation is ejected from the dispensing element. In some embodiments, at least about 90% of the formulation can pass through a circular opening having a diameter of between about 5 mm and 6 mm placed about 25 mm from where the formulation is ejected from the dispensing element. In some embodiments, at least about 95% of the formulation can pass through a circular opening having a diameter of between about 5 mm and 6 mm placed about 25 mm from where the formulation is ejected from the dispensing element.

In some embodiments, the formulation has a viscosity of between about 0.5 cP and about 100 cP. In some embodiments, the formulation has a viscosity of between about 0.5 cP and about 90 cP. In some embodiments, the formulation has a viscosity of between about 0.5 cP and about 80 cP. In some embodiments, the formulation has a viscosity of between about 0.5 cP and about 70 cP. In some embodiments, the formulation has a viscosity of between about 0.5 cP and about 60 cP. In some embodiments, the formulation has a viscosity of between about 0.5 cP and about 50 cP. In some embodiments, the formulation has a viscosity of between about 0.5 cP and about 40 cP. In some embodiments, the formulation has a viscosity of between about 0.5 cP and about 30 cP. In some embodiments, the formulation has a viscosity of between about 0.5 cP and about 20 cP. In some embodiments, the formulation has a viscosity of between about 0.5 cP and about 10 cP. In some embodiments, the formulation has a viscosity of between about 0.5 cP and about 5 cP. In some embodiments, the formulation has a viscosity of between about 5 cP and about 10 cP. In some embodiments, the formulation has a viscosity of between about 10 cP and about 15 cP. In some embodiments, the formulation has a viscosity of between about 15 cP and about 20 cP. In some embodiments, the formulation has a viscosity of between about 20 cP and about 25 cP. In some embodiments, the formulation has a viscosity of between about 25 cP and about 30 cP. In some embodiments, the formulation has a viscosity of between about 30 cP and about 35 cP. In some embodiments, the formulation has a viscosity of between about 35 cP and about 40 cP. In some embodiments, the formulation has a viscosity of between about 40 cP and about 45 cP. In some embodiments, the formulation has a viscosity of between about 45 cP and about 50 cP. In some embodiments, the formulation has a viscosity of between about 50 cP and about 55 cP. In some embodiments, the formulation has a viscosity of between about 55 cP and about 60 cP. In some embodiments, the formulation has a viscosity of between about 60 cP and about 65 cP. In some embodiments, the formulation has a viscosity of between about 65 cP and about 70 cP. In some embodiments, the formulation has a viscosity of between about 70 cP and about 75 cP. In some embodiments, the formulation has a viscosity of between about 75 cP and about 80 cP. In some embodiments, the formulation has a viscosity of between about 85 cP and about 90 cP. In some embodiments, the formulation has a viscosity of between about 95 cP and about 100 cP. In some embodiments, the formulation has a viscosity of higher than 100 cP. In some embodiments, the formulation has a viscosity of between about 45 cP and about 55 cP. In some embodiments, the formulation has a viscosity of between about 48 cP and about 52 cP. In some embodiments, the formulation has a viscosity of about 1 cP. In some embodiments, the formulation has a viscosity of about 50 cP.

In some embodiments, the formulation is ejected from the dispensing element at a velocity of between about 1 m/s and about 30 m/s. In some embodiments, the formulation is ejected from the dispensing element at a velocity of between about 2 m/s and about 4 m/s. In some embodiments, the formulation is ejected from the dispensing element at a velocity of between about 25 m/s and about 30 m/s. In some embodiments, the formulation is ejected from the dispensing element at a velocity. In some embodiments, the velocity is about 1 m/s. In some embodiments, the velocity is about 2 m/s. In some embodiments, the velocity is about 3 m/s. In some embodiments, the velocity is about 4 m/s. In some embodiments, the velocity is about 5 m/s. In some embodiments, the velocity is about 6 m/s. In some embodiments, the velocity is about 7 m/s. In some embodiments, the velocity is about 8 m/s. In some embodiments, the velocity is about 9 m/s. In some embodiments, the velocity is about 10 m/s. In some embodiments, the velocity is about 11 m/s. In some embodiments, the velocity is about 12 m/s. In some embodiments, the velocity is about 13 m/s. In some embodiments, the velocity is about 14 m/s. In some embodiments, the velocity is about 15 m/s. In some embodiments, the velocity is about 16 m/s. In some embodiments, the velocity is about 17 m/s. In some embodiments, the velocity is about 18 m/s. In some embodiments, the velocity is about 19 m/s. In some embodiments, the velocity is about 20 m/s. In some embodiments, the velocity is about 21 m/s. In some embodiments, the velocity is about 22 m/s. In some embodiments, the velocity is about 23 m/s. In some embodiments, the velocity is about 24 m/s. In some embodiments, the velocity is about 25 m/s. In some embodiments, the velocity is about 26 m/s. In some embodiments, the velocity is about 27 m/s. In some embodiments, the velocity is about 28 m/s. In some embodiments, the velocity is about 29 m/s. In some embodiments, the velocity is about 30 m/s. In some embodiments, the velocity is between about 1 m/s and about 2 m/s. In some embodiments, the velocity is between about 4 m/s and about 6 m/s. In some embodiments, the velocity is between about 6 m/s and about 8 m/s. In some embodiments, the velocity is between about 8 m/s and about 10 m/s. In some embodiments, the velocity is between about 10 m/s and about 12 m/s. In some embodiments, the velocity is between about 12 m/s and about 14 m/s. In some embodiments, the velocity is between about 14 m/s and about 16 m/s. In some embodiments, the velocity is between about 16 m/s and about 18 m/s. In some embodiments, the velocity is between about 18 m/s and about 20 m/s. In some embodiments, the velocity is between about 20 m/s and about 22 m/s. In some embodiments, the velocity is between about 22 m/s and about 24 m/s. In some embodiments, the velocity is between about 24 m/s and about 26 m/s. In some embodiments, the velocity is between about 26 m/s and about 28 m/s. In some embodiments, the velocity is between about 28 m/s and about 30 m/s.

In some embodiments, the formulation is ejected from a dispensing element, wherein at least about 75% of the formulation passes through a circular opening having a diameter of between about 5 mm and about 6 mm placed about 25 mm from where the formulation is ejected from the dispensing element. In some embodiments, the formulation has a viscosity of about 1 cP and is ejected from the dispensing element at a velocity of between about 2 m/s and about 4 m/s. In some embodiments, the formulation has a viscosity of about 50 cP and is ejected from the dispensing element at a velocity of between about 25 m/s and about 30 m/s. In some embodiments, the formulation has a viscosity of between about 0.5 cP and about 50 cP. In some embodiments, the formulation is ejected from the dispensing element at a velocity of between about 1 m/s and about 30 m/s.

The formulation can be delivered as a liquid jet or laminar flow. The formulation can be delivered as a flow having a Reynold's number of 2300 or less.

Laminar flow can refer to a smooth, regular movement of fluids where adjacent layers of particles experience relatively little mixing.

In some embodiments, the formulation is delivered as a liquid jet.

In some embodiments, the formulation is delivered as a laminar flow.

In some embodiments, delivering the formulation as a laminar flow increases on target delivery of the composition to the target region compared to delivering the formulation with a spray ejection profile. In some embodiments, delivering the formulation as a laminar flow decreases off target delivery of the composition to the nasal cavity compared to delivering the formulation with a spray ejection profile. In some embodiments, delivering the formulation as a laminar flow increases on target delivery of the composition to the target region and decreases off target delivery of the composition to the nasal cavity compared to delivering the formulation with a spray ejection profile.

In some embodiments, the formulation is delivered as a liquid jet.

In some embodiments, delivering the formulation as a liquid jet increases on target delivery of the composition to the target region compared to delivering the formulation with a spray ejection profile. In some embodiments, delivering the formulation as a liquid jet decreases off target delivery of the composition to the nasal cavity compared to delivering the formulation with a spray ejection profile. In some embodiments, delivering the formulation as a liquid jet increases on target delivery of the composition to the target region and decreases off target delivery of the composition to the nasal cavity compared to delivering the formulation with a spray ejection profile.

In some embodiments, the formulation is delivered as a flow having a Reynold's number of 2300 or less. In some embodiments, the formulation is delivered as a flow having a Reynold's number of 2200 or less. In some embodiments, the formulation is delivered as a flow having a Reynold's number of 2100 or less. In some embodiments, the formulation is delivered as a flow having a Reynold's number of 2000 or less. In some embodiments, the formulation is delivered as a flow having a Reynold's number of 1900 or less. In some embodiments, the formulation is delivered as a flow having a Reynold's number of 1800 or less. In some embodiments, the formulation is delivered as a flow having a Reynold's number of 1700 or less. In some embodiments, the formulation is delivered as a flow having a Reynold's number of 1600 or less. In some embodiments, the formulation is delivered as a flow having a Reynold's number of 1500 or less. In some embodiments, the formulation is delivered as a flow having a Reynold's number of 1400 or less. In some embodiments, the formulation is delivered as a flow having a Reynold's number of 1300 or less. In some embodiments, the formulation is delivered as a flow having a Reynold's number of 1200 or less. In some embodiments, the formulation is delivered as a flow having a Reynold's number of 1100 or less. In some embodiments, the formulation is delivered as a flow having a Reynold's number of 1000 or less. In some embodiments, the formulation is delivered as a flow having a Reynold's number of 900 or less. In some embodiments, the formulation is delivered as a flow having a Reynold's number of 800 or less. In some embodiments, the formulation is delivered as a flow having a Reynold's number of 700 or less. In some embodiments, the formulation is delivered as a flow having a Reynold's number of 600 or less. In some embodiments, the formulation is delivered as a flow having a Reynold's number of 500 or less. In some embodiments, the formulation is delivered as a flow having a Reynold's number of 400 or less. In some embodiments, the formulation is delivered as a flow having a Reynold's number of 300 or less.

In some embodiments, delivering the formulation as a flow having a Reynold's number of 2300 or less increases on target delivery of the composition to the target region compared to delivering the formulation with a spray ejection profile. In some embodiments, delivering the formulation as a flow having a Reynold's number of 2300 or less decreases off target delivery of the composition to the nasal cavity compared to delivering the formulation with a spray ejection profile. In some embodiments, delivering the formulation as a flow having a Reynold's number of 2300 or less increases on target delivery of the composition to the target region and decreases off target delivery of the composition to the nasal cavity compared to delivering the formulation with a spray ejection profile.

As shown herein, delivery of the therapeutic psychedelic by the method disclosed herein can result in a plasma concentration of the therapeutic psychedelic for a period of time after delivery. In some embodiments, the period of time is 5 min after delivery. In some embodiments, the period of time is 15 min after delivery. In some embodiments, the period of time is 30 min after delivery. In some embodiments, the period of time is 60 min after delivery. In some embodiments, the period of time is 90 min after delivery. In some embodiments, the period of time is 120 min after delivery. In some embodiments, the period of time is 150 min after delivery. In some embodiments, the period of time is 180 min after delivery. In some embodiments, the period of time is 200 min after delivery. In some embodiments, the period of time is 250 min after delivery.

In some embodiments, delivery of the therapeutic psychedelic to the olfactory region of the subject results in a plasma concentration of the therapeutic psychedelic about 15 min after delivery that is at least about 2 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 3 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 4 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 5 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 6 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 7 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 8 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 9 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 10 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 11 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 12 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 13 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 14 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is at least about 15 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 15 min after delivery that is more than about 15 times of a plasma concentration of the therapeutic psychedelic when about the same dose is delivered intravenously.

In some embodiments, delivery of the therapeutic psychedelic to the olfactory region of the subject results in a plasma concentration of the therapeutic psychedelic about 30 min after delivery that is at least about 2 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 3 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 4 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 5 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 6 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 7 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 8 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 9 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 10 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 11 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 12 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 13 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 14 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 15 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 16 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 17 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 18 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 19 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that is at least about 20 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 30 min after delivery that more than about 20 times of a plasma concentration of the therapeutic psychedelic when about the same dose is delivered intravenously.

In some embodiments, delivery of the therapeutic psychedelic to the olfactory region of the subject results in a plasma concentration of the therapeutic psychedelic about 60 min after delivery that is at least about 110% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 120% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 130% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 140% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 150% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 160% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 170% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 180% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 190% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 2 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 3 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 4 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 5 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 6 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 7 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 8 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 9 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is at least about 10 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 60 min after delivery that is more than about 10 times of a plasma concentration of the therapeutic psychedelic when about the same dose is delivered intravenously.

In some embodiments, delivery of the therapeutic psychedelic to the olfactory region of the subject results in a plasma concentration of the therapeutic psychedelic about 120 min after delivery that is at least about 110% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 120% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 130% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 140% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 150% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 160% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 170% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 180% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 190% of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 2 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 3 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 4 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 5 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 6 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 7 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 8 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 9 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is at least about 10 times of a plasma concentration of the therapeutic psychedelic when about the same dose is delivered intravenously. In some embodiments, delivery of the therapeutic psychedelic to the olfactory region of the subject results in a plasma concentration of the therapeutic psychedelic about 120 min after delivery that is more than about 10 times of a plasma concentration of the therapeutic psychedelic when about the same dose is delivered intravenously.

In some embodiments, selectively delivering the formulation to the olfactory region of the subject comprises inserting a dispensing element into an ejection zone of a nasal cavity of the subject.

In some embodiments, the method further comprises inserting a dispensing element into a nasal cavity of the subject.

In some embodiments, the dispensing element is positioned from about 0.1 mm to about 30 mm from the olfactory region or an anterior entry to the olfactory region. In some embodiments, the tip is positioned from about 0.1 mm to about 25 mm from the olfactory region or an anterior entry to the olfactory region. In some embodiments, the tip is positioned from about 0.1 mm to about 3 mm, about 0.1 mm to about 5 mm, about 0.1 mm to about 9 mm, about 0.1 mm to about 12 mm, about 0.1 mm to about 15 mm, about 0.1 mm to about 18 mm, about 0.1 mm to about 20 mm, about 0.1 mm to about 25 mm, about 0.1 mm to about 30 mm, about 3 mm to about 5 mm, about 3 mm to about 9 mm, about 3 mm to about 12 mm, about 3 mm to about 15 mm, about 3 mm to about 18 mm, about 3 mm to about 20 mm, about 3 mm to about 25 mm, about 3 mm to about 30 mm, about 5 mm to about 9 mm, about 5 mm to about 12 mm, about 5 mm to about 15 mm, about 5 mm to about 18 mm, about 5 mm to about 20 mm, about 5 mm to about 25 mm, about 5 mm to about 30 mm, about 9 mm to about 12 mm, about 9 mm to about 15 mm, about 9 mm to about 18 mm, about 9 mm to about 20 mm, about 9 mm to about 25 mm, about 9 mm to about 30 mm, about 12 mm to about 15 mm, about 12 mm to about 18 mm, about 12 mm to about 20 mm, about 12 mm to about 25 mm, about 12 mm to about 30 mm, about 15 mm to about 18 mm, about 15 mm to about 20 mm, about 15 mm to about 25 mm, about 15 mm to about 30 mm, about 18 mm to about 20 mm, about 18 mm to about 25 mm, about 18 mm to about 30 mm, about 20 mm to about 25 mm, about 20 mm to about 30 mm, or about 25 mm to about 30 mm, including increments therein. In some embodiments, the tip is positioned from about 0.1 mm, about 3 mm, about 5 mm, about 9 mm, about 12 mm, about 15 mm, about 18 mm, about 20 mm, about 25 mm, or about 30 mm from the olfactory region or an anterior entry to the olfactory region. In some embodiments, the tip is positioned from at least about 0.1 mm, about 3 mm, about 5 mm, about 9 mm, about 12 mm, about 15 mm, about 18 mm, about 20 mm, or about 25 mm from the olfactory region or an anterior entry to the olfactory region. In some embodiments, the dispensing element is positioned from at most about 3 mm, about 5 mm, about 9 mm, about 12 mm, about 15 mm, about 18 mm, about 20 mm, about 25 mm, or about 30 mm from the olfactory region or an anterior entry to the olfactory region.

In some embodiments, the method further comprises ejecting the formulation from an ejection zone of the nasal cavity.

In some embodiments, dispensing the formulation from the ejection zone increases on target delivery of the composition to the target region compared to dispensing the formulation outside the ejection zone. In some embodiments, dispensing the formulation from the ejection zone decreases off target delivery of the composition to the nasal cavity compared to dispensing the formulation outside the ejection zone. In some embodiments, dispensing the formulation from the ejection zone increases on target delivery of the composition to the target region and decreases off target delivery of the composition to the nasal cavity compared to dispensing the formulation outside the ejection zone.

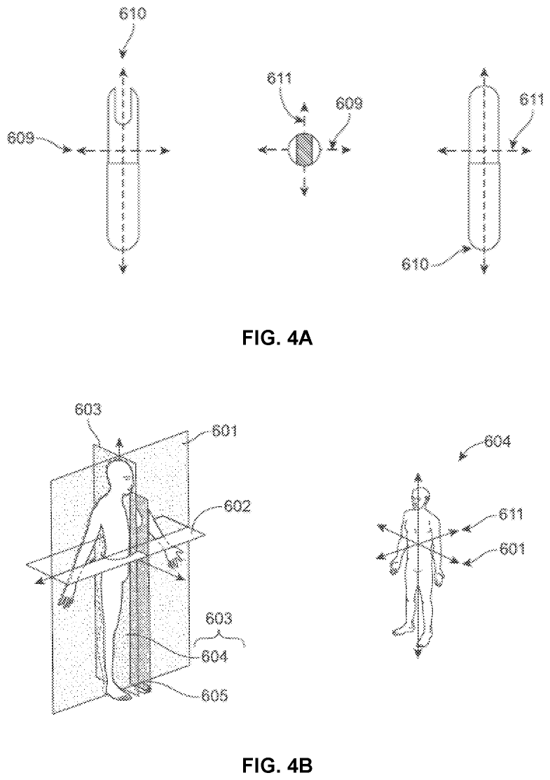

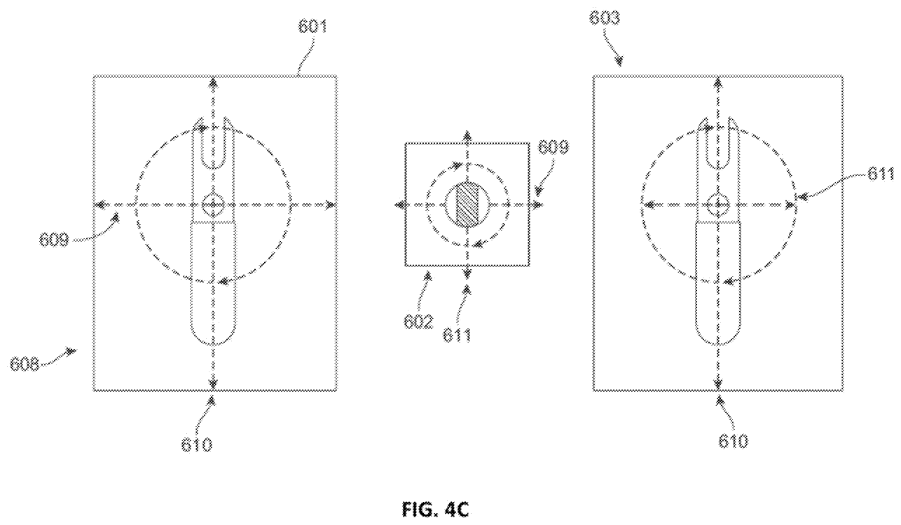

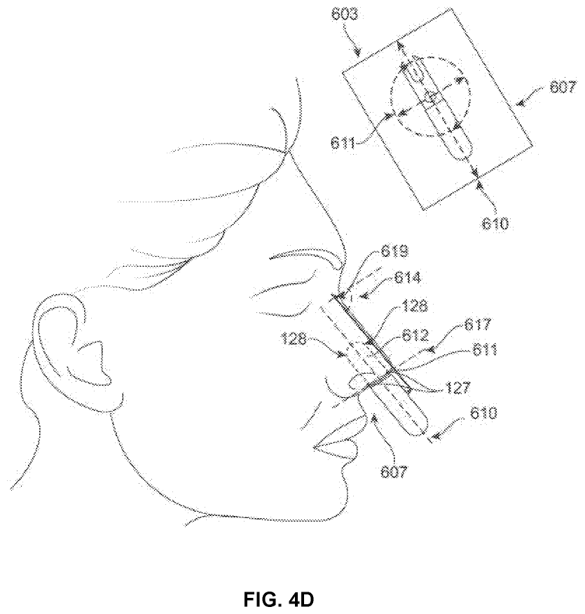

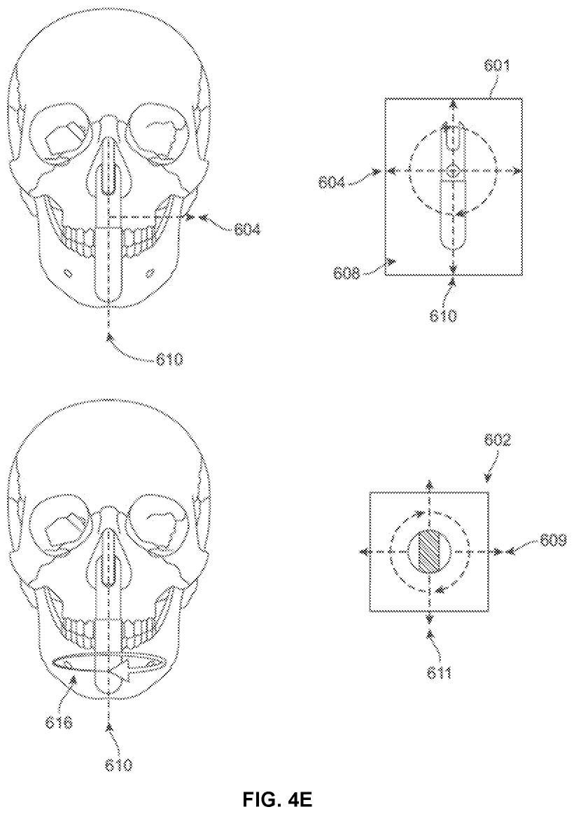

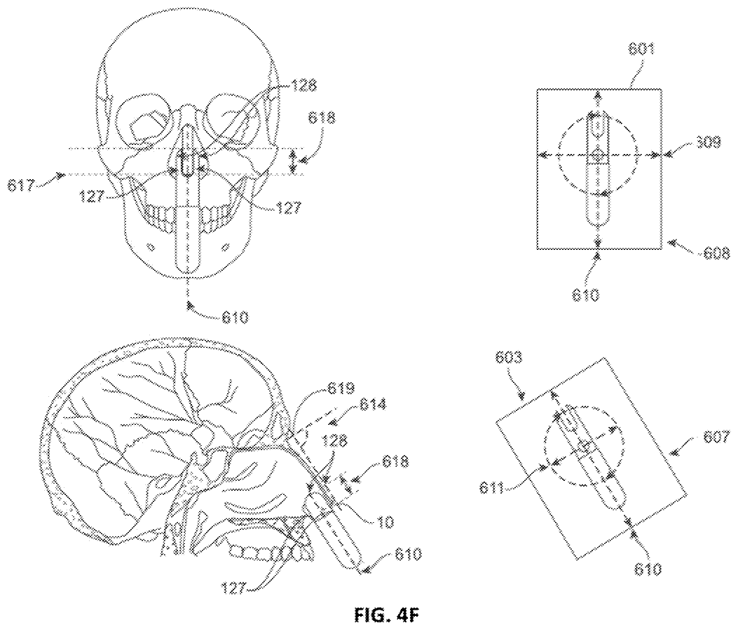

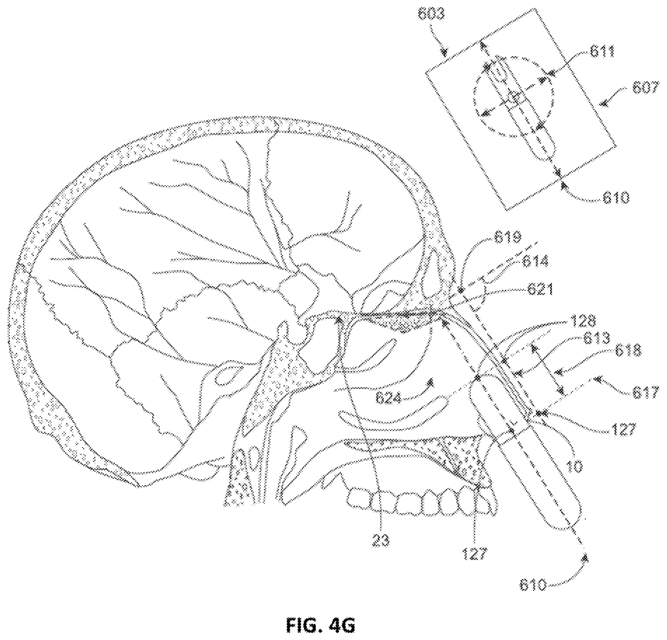

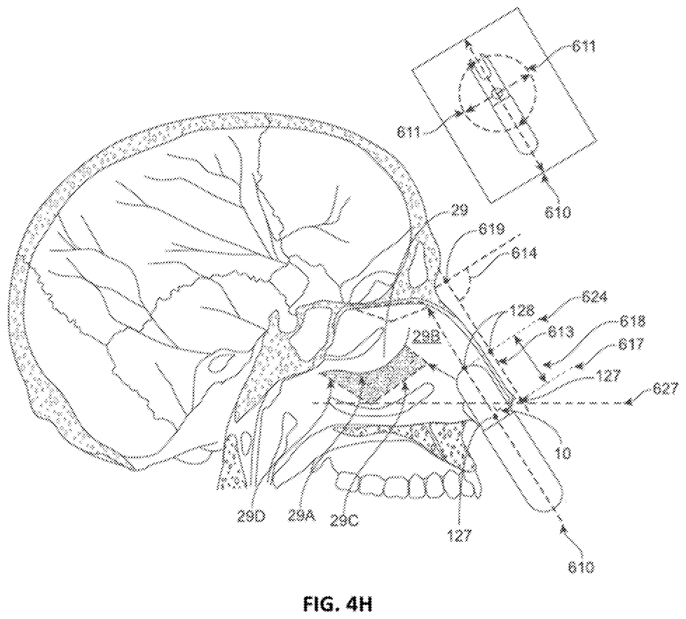

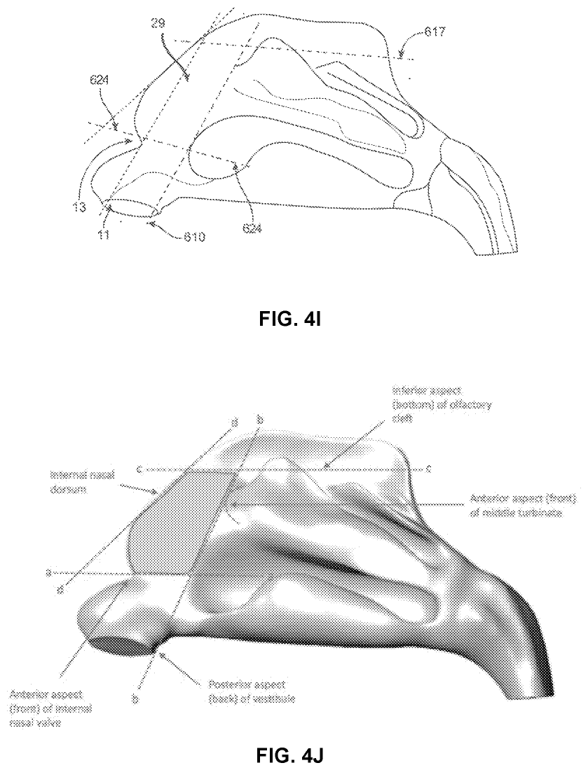

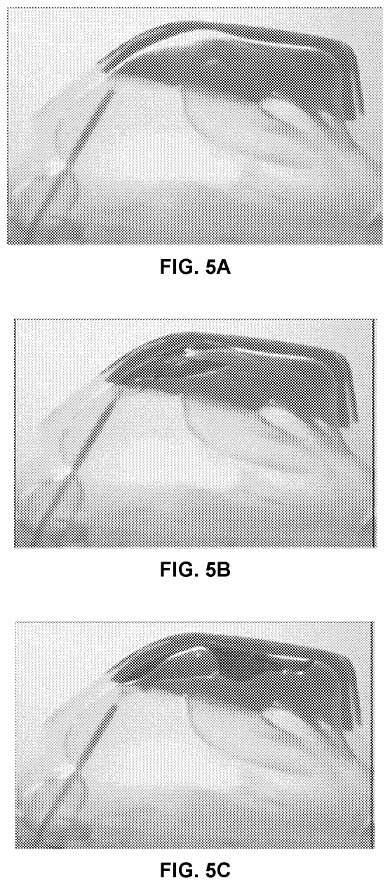

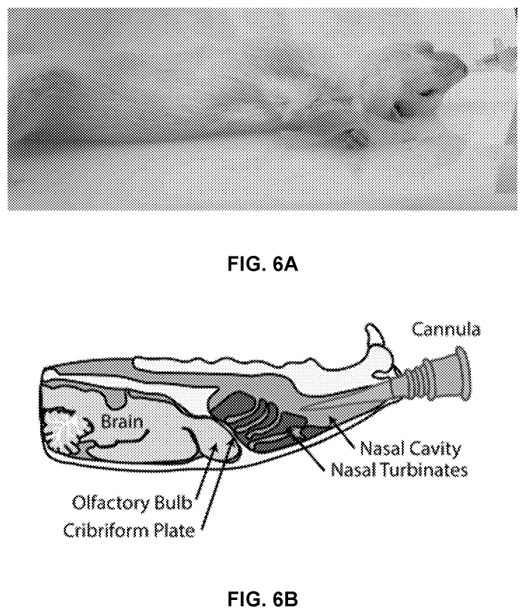

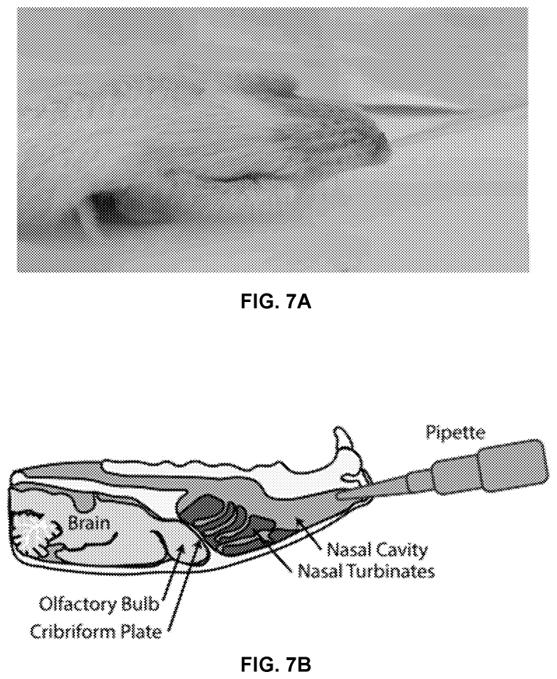

Referring to FIGS. 4A-4J. In some embodiments, the ejection zone 29 is: 0 mm to 30 mm superior to a horizontal line 627 that intersects the anterior aspect of the internal nasal valve 13, and 0 mm to 20 mm anterior to an inclined line 610 that intersects the anterior aspect of the middle turbinate 15 and the posterior aspect of the vestibule 21.

In some embodiments, the ejection zone 29 is further 0 mm to 40 mm inferior to a horizontal line 621 that is parallel to the inferior aspect of the olfactory cleft 23, 0 mm to 20 mm posterior to the internal nasal dorsum 622, 10 mm to 50 mm superior to a horizontal line 617 that intersects the inferior aspect of the columella 10, 0 mm to 30 mm superior to a horizontal line 624 that intersects the superior aspect of the inferior turbinate 16, 0 mm to 3 mm from the septum 24, or any combination thereof.

In some embodiments, the ejection zone 29 is a trapezium or irregular quadrilateral comprising (i) an inferior side 29A being a 10-25 mm line extending posteriorly and horizontally from the anterior aspect of the internal nasal valve 13, (ii) an anterior side 29B being a 10-35 mm line extending superiorly and parallel to the internal nasal dorsum 622 from the anterior aspect of the internal nasal valve 13, (iii) a superior side 29C being a 10-25 mm line extending posteriorly and horizontally from a point on the internal nasal dorsum 622 that is 0-10 mm inferior to the inferior aspect of the olfactory cleft 23, and (iv) a posterior line 29D being a 10-35 mm line extending vertically along a plane that intersects the anterior aspect of the middle turbinate 15.

In some embodiments, dispensing the formulation from the ejection zone 29 increases on target deposition of the formulation to an olfactory cleft 23.

In some embodiments, the subject engaging portion 106 engages a columella region 10 of the subject to seat a distal end 128 of the insertable portion 107 within an ejection zone 29 of a nasal cavity 11 of the subject, wherein the ejection zone 29 is: 0 mm to 30 mm superior to a horizontal line 627 that intersects the anterior aspect of the internal nasal valve 13, and 0 mm to 20 mm anterior to an inclined line 610 that intersects the anterior aspect of the middle turbinate 15 and the posterior aspect of the vestibule 21, and one or more of the following: 0 mm to 40 mm inferior to a horizontal line 621 that is parallel to the inferior aspect of the olfactory cleft 23, 0 mm to 20 mm posterior to the internal nasal dorsum 622, 10 mm to 50 mm superior to a horizontal line 623 that intersects the inferior aspect of the columella 10, 0 mm to 30 mm superior to a horizontal line 624 that intersects the superior aspect of the inferior turbinate 16, 0 mm to 3 mm from the septum 24 or any combination thereof.

In some embodiments, the ejection zone is superior to a horizontal line that intersects the anterior aspect of the internal nasal valve by about 1 mm to about 30 mm. In some embodiments, the ejection zone is superior to a horizontal line that intersects the anterior aspect of the internal nasal valve by about 1 mm to about 2 mm, about 1 mm to about 4 mm, about 1 mm to about 6 mm, about 1 mm to about 8 mm, about 1 mm to about 10 mm, about 1 mm to about 14 mm, about 1 mm to about 18 mm, about 1 mm to about 22 mm, about 1 mm to about 26 mm, about 1 mm to about 30 mm, about 2 mm to about 4 mm, about 2 mm to about 6 mm, about 2 mm to about 8 mm, about 2 mm to about 10 mm, about 2 mm to about 14 mm, about 2 mm to about 18 mm, about 2 mm to about 22 mm, about 2 mm to about 26 mm, about 2 mm to about 30 mm, about 4 mm to about 6 mm, about 4 mm to about 8 mm, about 4 mm to about 10 mm, about 4 mm to about 14 mm, about 4 mm to about 18 mm, about 4 mm to about 22 mm, about 4 mm to about 26 mm, about 4 mm to about 30 mm, about 6 mm to about 8 mm, about 6 mm to about 10 mm, about 6 mm to about 14 mm, about 6 mm to about 18 mm, about 6 mm to about 22 mm, about 6 mm to about 26 mm, about 6 mm to about 30 mm, about 8 mm to about 10 mm, about 8 mm to about 14 mm, about 8 mm to about 18 mm, about 8 mm to about 22 mm, about 8 mm to about 26 mm, about 8 mm to about 30 mm, about 10 mm to about 14 mm, about 10 mm to about 18 mm, about 10 mm to about 22 mm, about 10 mm to about 26 mm, about 10 mm to about 30 mm, about 14 mm to about 18 mm, about 14 mm to about 22 mm, about 14 mm to about 26 mm, about 14 mm to about 30 mm, about 18 mm to about 22 mm, about 18 mm to about 26 mm, about 18 mm to about 30 mm, about 22 mm to about 26 mm, about 22 mm to about 30 mm, or about 26 mm to about 30 mm, including increments therein. In some embodiments, the ejection zone is superior to a horizontal line that intersects the anterior aspect of the internal nasal valve by about 1 mm, about 2 mm, about 4 mm, about 6 mm, about 8 mm, about 10 mm, about 14 mm, about 18 mm, about 22 mm, about 26 mm, or about 30 mm. In some embodiments, the ejection zone is superior to a horizontal line that intersects the anterior aspect of the internal nasal valve by at least about 1 mm, about 2 mm, about 4 mm, about 6 mm, about 8 mm, about 10 mm, about 14 mm, about 18 mm, about 22 mm, or about 26 mm. In some embodiments, the ejection zone is superior to a horizontal line that intersects the anterior aspect of the internal nasal valve by at most about 2 mm, about 4 mm, about 6 mm, about 8 mm, about 10 mm, about 14 mm, about 18 mm, about 22 mm, about 26 mm, or about 30 mm. In some embodiments, the ejection zone is anterior to an inclined line that intersects the anterior aspect of the middle turbinate and the posterior aspect of the vestibule by about 1 mm to about 20 mm. In some embodiments, the ejection zone is anterior to an inclined line that intersects the anterior aspect of the middle turbinate and the posterior aspect of the vestibule by about 1 mm to about 2 mm, about 1 mm to about 4 mm, about 1 mm to about 6 mm, about 1 mm to about 8 mm, about 1 mm to about 10 mm, about 1 mm to about 12 mm, about 1 mm to about 14 mm, about 1 mm to about 16 mm, about 1 mm to about 18 mm, about 1 mm to about 20 mm, about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or about 18 mm to about 20 mm, including increments therein. In some embodiments, the ejection zone is anterior to an inclined line that intersects the anterior aspect of the middle turbinate and the posterior aspect of the vestibule by about 1 mm, about 2 mm, about 4 mm, about 6 mm, about 8 mm, about 10 mm, about 12 mm, about 14 mm, about 16 mm, about 18 mm, or about 20 mm. In some embodiments, the ejection zone is anterior to an inclined line that intersects the anterior aspect of the middle turbinate and the posterior aspect of the vestibule by at least about 1 mm, about 2 mm, about 4 mm, about 6 mm, about 8 mm, about 10 mm, about 12 mm, about 14 mm, about 16 mm, or about 18 mm. In some embodiments, the ejection zone is anterior to an inclined line that intersects the anterior aspect of the middle turbinate and the posterior aspect of the vestibule by at most about 2 mm, about 4 mm, about 6 mm, about 8 mm, about 10 mm, about 12 mm, about 14 mm, about 16 mm, about 18 mm, or about 20 mm. In some embodiments, the ejection zone is inferior to a horizontal line that is parallel to the inferior aspect of the olfactory cleft by about 1 mm to about 35 mm. In some embodiments, the ejection zone is inferior to a horizontal line that is parallel to the inferior aspect of the olfactory cleft by about 1 mm to about 2 mm, about 1 mm to about 3 mm, about 1 mm to about 4 mm, about 1 mm to about 5 mm, about 1 mm to about 8 mm, about 1 mm to about 12 mm, about 1 mm to about 15 mm, about 1 mm to about 18 mm, about 1 mm to about 21 mm, about 1 mm to about 25 mm, about 1 mm to about 30 mm, about 1 mm to about 35 mm, about 2 mm to about 3 mm, about 2 mm to about 4 mm, about 2 mm to about 5 mm, about 2 mm to about 8 mm, about 2 mm to about 12 mm, about 2 mm to about 15 mm, about 2 mm to about 18 mm, about 2 mm to about 21 mm, about 2 mm to about 25 mm, about 2 mm to about 30 mm, about 2 mm to about 35 mm, about 3 mm to about 4 mm, about 3 mm to about 5 mm, about 3 mm to about 8 mm, about 3 mm to about 12 mm, about 3 mm to about 15 mm, about 3 mm to about 18 mm, about 3 mm to about 21 mm, about 3 mm to about 25 mm, about 3 mm to about 30 mm, about 3 mm to about 35 mm, about 4 mm to about 5 mm, about 4 mm to about 8 mm, about 4 mm to about 12 mm, about 4 mm to about 15 mm, about 4 mm to about 18 mm, about 4 mm to about 21 mm, about 4 mm to about 25 mm, about 4 mm to about 30 mm, about 4 mm to about 35 mm, about 5 mm to about 8 mm, about 5 mm to about 12 mm, about 5 mm to about 15 mm, about 5 mm to about 18 mm, about 5 mm to about 21 mm, about 5 mm to about 25 mm, about 5 mm to about 30 mm, about 5 mm to about 35 mm, about 8 mm to about 15 mm, about 8 mm to about 18 mm, about 8 mm to about 21 mm, about 8 mm to about 25 mm, about 8 mm to about 30 mm, about 8 mm to about 35 mm, about 12 mm to about 15 mm, about 12 mm to about 18 mm, about 12 mm to about 21 mm, about 12 mm to about 25 mm, about 12 mm to about 30 mm, about 12 mm to about 35 mm, about 15 mm to about 18 mm, about 15 mm to about 21 mm, about 15 mm to about 25 mm, about 15 mm to about 30 mm, about 15 mm to about 35 mm, about 18 mm to about 21 mm, about 18 mm to about 25 mm, about 18 mm to about 30 mm, about 18 mm to about 35 mm, about 21 mm to about 25 mm, about 21 mm to about 30 mm, about 21 mm to about 35 mm, about 25 mm to about 30 mm, about 25 mm to about 35 mm, or about 30 mm to about 35 mm, including increments therein. In some embodiments, the ejection zone is inferior to a horizontal line that is parallel to the inferior aspect of the olfactory cleft by about 1 mm, about 2 mm, about 3 mm, about 4 mm, about 5 mm, about 8 mm, about 12 mm, about 15 mm, about 18 mm, about 21 mm, about 25 mm, about 30 mm, or about 35 mm. In some embodiments, the ejection zone is inferior to a horizontal line that is parallel to the inferior aspect of the olfactory cleft by at least about 1 mm, about 2 mm, about 3 mm, about 4 mm, about 5 mm, about 8 mm, about 12 mm, about 15 mm, about 18 mm, about 21 mm, about 25 mm, or about 30 mm. In some embodiments, the ejection zone is inferior to a horizontal line that is parallel to the inferior aspect of the olfactory cleft by at most about 2 mm, about 3 mm, about 4 mm, about 5 mm, about 8 mm, about 12 mm, about 15 mm, about 18 mm, about 21 mm, about 25 mm, about 30 mm, or about 35 mm. In some embodiments, the ejection zone is posterior to the internal nasal dorsum by about 1 mm to about 20 mm. In some embodiments, the ejection zone is posterior to the internal nasal dorsum by about 1 mm to about 2 mm, about 1 mm to about 3 mm, about 1 mm to about 4 mm, about 1 mm to about 6 mm, about 1 mm to about 8 mm, about 1 mm to about 10 mm, about 1 mm to about 12 mm, about 1 mm to about 14 mm, about 1 mm to about 16 mm, about 1 mm to about 18 mm, about 1 mm to about 20 mm, about 2 mm to about 3 mm, about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 3 mm to about 4 mm, about 3 mm to about 6 mm, about 3 mm to about 8 mm, about 3 mm to about 10 mm, about 3 mm to about 12 mm, about 3 mm to about 14 mm, about 3 mm to about 16 mm, about 3 mm to about 18 mm, about 3 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or about 18 mm to about 20 mm, including increments therein. In some embodiments, the ejection zone is posterior to the internal nasal dorsum by about 1 mm, about 2 mm, about 3 mm, about 4 mm, about 6 mm, about 8 mm, about 10 mm, about 12 mm, about 14 mm, about 16 mm, about 18 mm, or about 20 mm. In some embodiments, the ejection zone is posterior to the internal nasal dorsum by at least about 1 mm, about 2 mm, about 3 mm, about 4 mm, about 6 mm, about 8 mm, about 10 mm, about 12 mm, about 14 mm, about 16 mm, or about 18 mm. In some embodiments, the ejection zone is posterior to the internal nasal dorsum by at most about 2 mm, about 3 mm, about 4 mm, about 6 mm, about 8 mm, about 10 mm, about 12 mm, about 14 mm, about 16 mm, about 18 mm, or about 20 mm. In some embodiments, the ejection zone is superior to a horizontal line that intersects the inferior aspect of the columella by about 10 mm to about 50 mm. In some embodiments, the ejection zone is superior to a horizontal line that intersects the inferior aspect of the columella by about 10 mm to about 12 mm, about 10 mm to about 16 mm, about 10 mm to about 20 mm, about 10 mm to about 24 mm, about 10 mm to about 28 mm, about 10 mm to about 32 mm, about 10 mm to about 36 mm, about 10 mm to about 40 mm, about 10 mm to about 45 mm, about 10 mm to about 50 mm, about 12 mm to about 16 mm, about 12 mm to about 20 mm, about 12 mm to about 24 mm, about 12 mm to about 28 mm, about 12 mm to about 32 mm, about 12 mm to about 36 mm, about 12 mm to about 40 mm, about 12 mm to about 45 mm, about 12 mm to about 50 mm, about 16 mm to about 20 mm, about 16 mm to about 24 mm, about 16 mm to about 28 mm, about 16 mm to about 32 mm, about 16 mm to about 36 mm, about 16 mm to about 40 mm, about 16 mm to about 45 mm, about 16 mm to about 50 mm, about 20 mm to about 24 mm, about 20 mm to about 28 mm, about 20 mm to about 32 mm, about 20 mm to about 36 mm, about 20 mm to about 40 mm, about 20 mm to about 45 mm, about 20 mm to about 50 mm, about 24 mm to about 28 mm, about 24 mm to about 32 mm, about 24 mm to about 36 mm, about 24 mm to about 40 mm, about 24 mm to about 45 mm, about 24 mm to about 50 mm, about 28 mm to about 32 mm, about 28 mm to about 36 mm, about 28 mm to about 40 mm, about 28 mm to about 45 mm, about 28 mm to about 50 mm, about 32 mm to about 36 mm, about 32 mm to about 40 mm, about 32 mm to about 45 mm, about 32 mm to about 50 mm, about 36 mm to about 40 mm, about 36 mm to about 45 mm, about 36 mm to about 50 mm, about 40 mm to about 45 mm, about 40 mm to about 50 mm, or about 45 mm to about 50 mm, including increments therein. In some embodiments, the ejection zone is superior to a horizontal line that intersects the inferior aspect of the columella by about 10 mm, about 12 mm, about 16 mm, about 20 mm, about 24 mm, about 28 mm, about 32 mm, about 36 mm, about 40 mm, about 45 mm, or about 50 mm. In some embodiments, the ejection zone is superior to a horizontal line that intersects the inferior aspect of the columella by at least about 10 mm, about 12 mm, about 16 mm, about 20 mm, about 24 mm, about 28 mm, about 32 mm, about 36 mm, about 40 mm, or about 45 mm. In some embodiments, the ejection zone is superior to a horizontal line that intersects the inferior aspect of the columella by at most about 12 mm, about 16 mm, about 20 mm, about 24 mm, about 28 mm, about 32 mm, about 36 mm, about 40 mm, about 45 mm, or about 50 mm. In some embodiments, the ejection zone is superior to a horizontal line that intersects the superior aspect of the inferior turbinate by about 1 mm to about 30 mm. In some embodiments, the ejection zone is superior to a horizontal line that intersects the superior aspect of the inferior turbinate by about 1 mm to about 2 mm, about 1 mm to about 4 mm, about 1 mm to about 6 mm, about 1 mm to about 8 mm, about 1 mm to about 10 mm, about 1 mm to about 12 mm, about 1 mm to about 15 mm, about 1 mm to about 18 mm, about 1 mm to about 21 mm, about 1 mm to about 25 mm, about 1 mm to about 30 mm, about 2 mm to about 4 mm, about 2 mm to about 6 mm, about 2 mm to about 8 mm, about 2 mm to about 10 mm, about 2 mm to about 12 mm, about 2 mm to about 15 mm, about 2 mm to about 18 mm, about 2 mm to about 21 mm, about 2 mm to about 25 mm, about 2 mm to about 30 mm, about 4 mm to about 6 mm, about 4 mm to about 8 mm, about 4 mm to about 10 mm, about 4 mm to about 12 mm, about 4 mm to about 15 mm, about 4 mm to about 18 mm, about 4 mm to about 21 mm, about 4 mm to about 25 mm, about 4 mm to about 30 mm, about 6 mm to about 8 mm, about 6 mm to about 10 mm, about 6 mm to about 12 mm, about 6 mm to about 15 mm, about 6 mm to about 18 mm, about 6 mm to about 21 mm, about 6 mm to about 25 mm, about 6 mm to about 30 mm, about 8 mm to about 10 mm, about 8 mm to about 12 mm, about 8 mm to about 15 mm, about 8 mm to about 18 mm, about 8 mm to about 21 mm, about 8 mm to about 25 mm, about 8 mm to about 30 mm, about 10 mm to about 12 mm, about 10 mm to about 15 mm, about 10 mm to about 18 mm, about 10 mm to about 21 mm, about 10 mm to about 25 mm, about 10 mm to about 30 mm, about 12 mm to about 15 mm, about 12 mm to about 18 mm, about 12 mm to about 21 mm, about 12 mm to about 25 mm, about 12 mm to about 30 mm, about 15 mm to about 18 mm, about 15 mm to about 21 mm, about 15 mm to about 25 mm, about 15 mm to about 30 mm, about 18 mm to about 21 mm, about 18 mm to about 25 mm, about 18 mm to about 30 mm, about 21 mm to about 25 mm, about 21 mm to about 30 mm, or about 25 mm to about 30 mm, including increments therein. In some embodiments, the ejection zone is superior to a horizontal line that intersects the superior aspect of the inferior turbinate by about 1 mm, about 2 mm, about 4 mm, about 6 mm, about 8 mm, about 10 mm, about 12 mm, about 15 mm, about 18 mm, about 21 mm, about 25 mm, or about 30 mm. In some embodiments, the ejection zone is superior to a horizontal line that intersects the superior aspect of the inferior turbinate by at least about 1 mm, about 2 mm, about 4 mm, about 6 mm, about 8 mm, about 10 mm, about 12 mm, about 15 mm, about 18 mm, about 21 mm, or about 25 mm. In some embodiments, the ejection zone is superior to a horizontal line that intersects the superior aspect of the inferior turbinate by at most about 2 mm, about 4 mm, about 6 mm, about 8 mm, about 10 mm, about 12 mm, about 15 mm, about 18 mm, about 21 mm, about 25 mm, or about 30 mm. In some embodiments, the ejection zone is away from the septum by about 0.1 mm to about 3 mm. In some embodiments, the ejection zone is away from the septum by about 0.1 mm to about 0.2 mm, about 0.1 mm to about 0.4 mm, about 0.1 mm to about 0.6 mm, about 0.1 mm to about 0.8 mm, about 0.1 mm to about 1 mm, about 0.1 mm to about 1.25 mm, about 0.1 mm to about 1.5 mm, about 0.1 mm to about 1.75 mm, about 0.1 mm to about 2 mm, about 0.1 mm to about 2.5 mm, about 0.1 mm to about 3 mm, about 0.2 mm to about 0.4 mm, about 0.2 mm to about 0.6 mm, about 0.2 mm to about 0.8 mm, about 0.2 mm to about 1 mm, about 0.2 mm to about 1.25 mm, about 0.2 mm to about 1.5 mm, about 0.2 mm to about 1.75 mm, about 0.2 mm to about 2 mm, about 0.2 mm to about 2.5 mm, about 0.2 mm to about 3 mm, about 0.4 mm to about 0.6 mm, about 0.4 mm to about 0.8 mm, about 0.4 mm to about 1 mm, about 0.4 mm to about 1.25 mm, about 0.4 mm to about 1.5 mm, about 0.4 mm to about 1.75 mm, about 0.4 mm to about 2 mm, about 0.4 mm to about 2.5 mm, about 0.4 mm to about 3 mm, about 0.6 mm to about 0.8 mm, about 0.6 mm to about 1 mm, about 0.6 mm to about 1.25 mm, about 0.6 mm to about 1.5 mm, about 0.6 mm to about 1.75 mm, about 0.6 mm to about 2 mm, about 0.6 mm to about 2.5 mm, about 0.6 mm to about 3 mm, about 0.8 mm to about 1 mm, about 0.8 mm to about 1.25 mm, about 0.8 mm to about 1.5 mm, about 0.8 mm to about 1.75 mm, about 0.8 mm to about 2 mm, about 0.8 mm to about 2.5 mm, about 0.8 mm to about 3 mm, about 1 mm to about 1.25 mm, about 1 mm to about 1.5 mm, about 1 mm to about 1.75 mm, about 1 mm to about 2 mm, about 1 mm to about 2.5 mm, about 1 mm to about 3 mm, about 1.25 mm to about 1.5 mm, about 1.25 mm to about 1.75 mm, about 1.25 mm to about 2 mm, about 1.25 mm to about 2.5 mm, about 1.25 mm to about 3 mm, about 1.5 mm to about 1.75 mm, about 1.5 mm to about 2 mm, about 1.5 mm to about 2.5 mm, about 1.5 mm to about 3 mm, about 1.75 mm to about 2 mm, about 1.75 mm to about 2.5 mm, about 1.75 mm to about 3 mm, about 2 mm to about 2.5 mm, about 2 mm to about 3 mm, or about 2.5 mm to about 3 mm, including increments therein. In some embodiments, the ejection zone is away from the septum by about 0.1 mm, about 0.2 mm, about 0.4 mm, about 0.6 mm, about 0.8 mm, about 1 mm, about 1.25 mm, about 1.5 mm, about 1.75 mm, about 2 mm, about 2.5 mm, or about 3 mm. In some embodiments, the ejection zone is away from the septum by at least about 0.1 mm, about 0.2 mm, about 0.4 mm, about 0.6 mm, about 0.8 mm, about 1 mm, about 1.25 mm, about 1.5 mm, about 1.75 mm, about 2 mm, or about 2.5 mm. In some embodiments, the ejection zone is away from the septum by at most about 0.2 mm, about 0.4 mm, about 0.6 mm, about 0.8 mm, about 1 mm, about 1.25 mm, about 1.5 mm, about 1.75 mm, about 2 mm, about 2.5 mm, or about 3 mm.

The dispensing element can be a dispensing element of a device for olfactory delivery of the therapeutic psychedelic. The device can be inserted into the nasal cavity of the subject for delivery. The device can be inserted at an angle from a vertical line. In some embodiments, the vertical line is a line perpendicular to a horizontal line that is parallel to the inferior aspect of the olfactory cleft, as depicted as line c-c of FIG. 4J.

In some embodiments, the device is inserted into the nasal cavity of the subject at an angle of between about 30 degrees to about 40 degrees from a vertical line, the vertical line being perpendicular to a horizontal line that is parallel to an inferior aspect of the olfactory cleft of the subject. In some embodiments, the angle is about 30 degrees from the vertical line. In some embodiments, the angle is about 31 degrees from the vertical line. In some embodiments, the angle is about 32 degrees from the vertical line. In some embodiments, the angle is about 33 degrees from the vertical line. In some embodiments, the angle is about 34 degrees from the vertical line. In some embodiments, the angle is about 35 degrees from the vertical line. In some embodiments, the angle is about 36 degrees from the vertical line. In some embodiments, the angle is about 37 degrees from the vertical line. In some embodiments, the angle is about 38 degrees from the vertical line. In some embodiments, the angle is about 39 degrees from the vertical line. In some embodiments, the angle is about 40 degrees from the vertical line.

In some embodiments, the formulation has a viscosity of about 1 cP. In some embodiments, delivering the formulation comprises ejecting the formulation from the ejection zone at a velocity of about 1.0 m/s. In some embodiments, delivering the formulation comprises ejecting the formulation from the ejection zone at a velocity of about 1.1 m/s. In some embodiments, delivering the formulation comprises ejecting the formulation from the ejection zone at a velocity of about 1.2 m/s. In some embodiments, delivering the formulation comprises ejecting the formulation from the ejection zone at a velocity of about 1.3 m/s. In some embodiments, delivering the formulation comprises ejecting the formulation from the ejection zone at a velocity of about 1.4 m/s. In some embodiments, delivering the formulation comprises ejecting the formulation from the ejection zone at a velocity of about 1.5 m/s. In some embodiments, delivering the formulation comprises ejecting the formulation from the ejection zone at a velocity of about 1.6 m/s. In some embodiments, delivering the formulation comprises ejecting the formulation from the ejection zone at a velocity of about 1.7 m/s. In some embodiments, delivering the formulation comprises ejecting the formulation from the ejection zone at a velocity of about 1.8 m/s. In some embodiments, delivering the formulation comprises ejecting the formulation from the ejection zone at a velocity of about 1.9 m/s. In some embodiments, delivering the formulation comprises ejecting the formulation from the ejection zone at a velocity of about 2.0 m/s. In some embodiments, delivering the formulation comprises ejecting the formulation from the ejection zone at a velocity of about 2.1 m/s. In some embodiments, delivering the formulation comprises ejecting the formulation from the ejection zone at a velocity of about 2.2 m/s. In some embodiments, delivering the formulation comprises ejecting the formulation from the ejection zone at a velocity of about 2.3 m/s. In some embodiments, delivering the formulation comprises ejecting the formulation from the ejection zone at a velocity of about 2.4 m/s. In some embodiments, delivering the formulation comprises ejecting the formulation from the ejection zone at a velocity of about 2.5 m/s. In some embodiments, delivering the formulation comprises ejecting the formulation n from the ejection zone at a velocity of about 2.6 m/s. In some embodiments, delivering the formulation comprises ejecting the formulation from the ejection zone at a velocity of about 2.7 m/s. In some embodiments, delivering the formulation comprises ejecting the formulation from the ejection zone at a velocity of about 2.8 m/s. In some embodiments, delivering the formulation comprises ejecting the formulation from the ejection zone at a velocity of about 2.9 m/s. In some embodiments, delivering the formulation comprises ejecting the formulation from the ejection zone at a velocity of between about 1.1 m/s and about 1.9 m/s. In some embodiments, delivering the formulation comprises ejecting the formulation from the ejection zone at a velocity of between about 1.0 m/s and about 2.2 m/s. In some embodiments, delivering the formulation comprises ejecting the formulation from the ejection zone at a velocity of between about 1.0 m/s and about 2.8 m/s.

In some embodiments, the formulation has a viscosity of about 50 cP. In some embodiments, delivering the formulation comprises ejecting the formulation from the ejection zone at a velocity of about 4 m/s. In some embodiments, the formulation has a viscosity of about 50 cP. In some embodiments, delivering the formulation comprises ejecting the formulation from the ejection zone at a velocity of about 4.7 m/s. In some embodiments, delivering the formulation comprises ejecting the formulation from the ejection zone at a velocity of about 5 m/s. In some embodiments, delivering the formulation comprises ejecting the formulation from the ejection zone at a velocity of about 6 m/s. In some embodiments, delivering the formulation comprises ejecting the formulation from the ejection zone at a velocity of about 7 m/s. In some embodiments, delivering the formulation comprises ejecting the formulation from the ejection zone at a velocity of about 8 m/s. In some embodiments, delivering the formulation comprises ejecting the formulation from the ejection zone at a velocity of about 9 m/s. In some embodiments, delivering the formulation comprises ejecting the formulation from the ejection zone at a velocity of about 10 m/s. In some embodiments, delivering the formulation comprises ejecting the formulation from the ejection zone at a velocity of about 11 m/s. In some embodiments, delivering the formulation comprises ejecting the formulation from the ejection zone at a velocity of about 12 m/s. In some embodiments, delivering the formulation comprises ejecting the formulation from the ejection zone at a velocity of about 13 m/s. In some embodiments, delivering the formulation comprises ejecting the formulation from the ejection zone at a velocity of about 14 m/s. In some embodiments, delivering the formulation comprises ejecting the formulation from the ejection zone at a velocity of about 15 m/s. In some embodiments, delivering the formulation comprises ejecting the formulation from the ejection zone at a velocity of about 16 m/s. In some embodiments, delivering the formulation comprises ejecting the formulation from the ejection zone at a velocity of about 17 m/s. In some embodiments, delivering the formulation comprises ejecting the formulation from the ejection zone at a velocity of about 18 m/s. In some embodiments, delivering the formulation comprises ejecting the formulation from the ejection zone at a velocity of about 18.6 m/s. In some embodiments, delivering the formulation comprises ejecting the formulation from the ejection zone at a velocity of about 19 m/s. In some embodiments, delivering the formulation comprises ejecting the formulation from the ejection zone at a velocity of between about 4.7 m/s and about 18.6 m/s. In some embodiments, delivering the formulation comprises ejecting the formulation from the ejection zone at a velocity of between about 13.9 m/s and about 15.8 m/s. In some embodiments, delivering the formulation comprises ejecting the formulation from the ejection zone at a velocity of between about 11.0 m/s and about 15.8 m/s.

As demonstrated herein, the formulation delivered by the method disclosed herein can remain at the olfactory cleft of the subject for a period of time longer than when the formulation is delivered by a nasal spray.

In some embodiments, the formulation delivered to the olfactory cleft of the subject remains at the olfactory cleft for at least about 10 seconds. In some embodiments, the formulation delivered to the olfactory cleft of the subject remains at the olfactory cleft for at least about 30 seconds. In some embodiments, the formulation delivered to the olfactory cleft of the subject remains at the olfactory cleft for at least about 1 min. In some embodiments, the formulation delivered to the olfactory cleft of the subject remains at the olfactory cleft for at least about 2 min. In some embodiments, the formulation delivered to the olfactory cleft of the subject remains at the olfactory cleft for at least about 3 min. In some embodiments, the formulation delivered to the olfactory cleft of the subject remains at the olfactory cleft for at least about 4 min. In some embodiments, the formulation delivered to the olfactory cleft of the subject remains at the olfactory cleft for at least about 5 min. In some embodiments, the formulation delivered to the olfactory cleft of the subject remains at the olfactory cleft for at least about 6 min. In some embodiments, the formulation delivered to the olfactory cleft of the subject remains at the olfactory cleft for at least about 7 min. In some embodiments, the formulation delivered to the olfactory cleft of the subject remains at the olfactory cleft for at least about 8 min. In some embodiments, the formulation delivered to the olfactory cleft of the subject remains at the olfactory cleft for at least about 9 min. In some embodiments, the formulation delivered to the olfactory cleft of the subject remains at the olfactory cleft for at least about 10 min. In some embodiments, the formulation delivered to the olfactory cleft of the subject remains at the olfactory cleft for at least about 11 min. In some embodiments, the formulation delivered to the olfactory cleft of the subject remains at the olfactory cleft for at least about 12 min. In some embodiments, the formulation delivered to the olfactory cleft of the subject remains at the olfactory cleft for at least about 13 min. In some embodiments, the formulation delivered to the olfactory cleft of the subject remains at the olfactory cleft for at least about 14 min. In some embodiments, the formulation delivered to the olfactory cleft of the subject remains at the olfactory cleft for at least about 15 min. In some embodiments, the formulation delivered to the olfactory cleft of the subject remains at the olfactory cleft for longer than 15 min. In some embodiments, the formulation delivered to the olfactory cleft of the subject remains at the olfactory cleft for a period of time longer than if the formulation was delivered by nasal spray.

Any suitable therapeutic psychedelics may be used with the methods disclosed herein.

The therapeutic psychedelic can be a modified psychedelic. The therapeutic psychedelics can be conjugated to an agent. The agent can be a therapeutic agent, such as a drug or a radioisotope.

In some embodiments, the therapeutic psychedelic is conjugated to a therapeutic agent. In some embodiments, the therapeutic agent is a drug. In some embodiments, the therapeutic agent is a radioisotope.

Examples of therapeutic psychedelics include but are not limited to: ketamine, R-ketamine, S-ketamine, psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, a serotonin antagonist, and derivatives and analogs thereof.

In some embodiments, the cannabinoid is selected from the group consisting of: cannabidiol, tetrahydrocannabinol, cannabinol, cannabigerol, cannabichromene, tetrahydrocannabivarin, cannabidivarin, and analogs and derivatives thereof such as acids and esters.

In some embodiments, the 5-HT2C receptor agonist is selected from the group consisting of lorcaserin, vabicaserin, and analogs and derivatives thereof.

In some embodiments, the dopamine antagonist is selected from the group consisting of: Chlorpromazine, Fluphenazine, Haloperidol, Loxapine, Molindone, Perphenazine, Pimozide, Prochlorperazine, Thiothixene, Thoridazine, Trifluoperazine, Aripiprazole, Asenapine, Brexpiprazole, Cariprazine, Clozapine, Iloperidone, Lumateperone, Lurasidone, Olanzapine, Quetiapine, Paliperidone, Pimavanserin, Risperidone, Ziprasidone, Amisulpride, Metoclopramide, Droperidol, Haloperidol, Chlorpromazine, Prochlorperazine, Olanzapine, and analogs and derivatives thereof.

In some embodiments, the therapeutic psychedelic comprises ketamine, or a derivative or analog thereof.

It is demonstrated herein that the method disclosed herein can result in delivery of the therapeutic psychedelic to a non-CNS target.

In some embodiments, the non-CNS target is blood.

The method disclosed herein is capable of delivering the therapeutic psychedelic to one or more of the group consisting of cerebrospinal fluid, the central nervous system, and a region, tissue, or organ thereof. In some embodiments, at least about 5% of the therapeutic psychedelic in the formulation is delivered to one or more of the group consisting of cerebrospinal fluid, the central nervous system, and a region, tissue, or organ thereof. In some embodiments, at least about 10% of the therapeutic psychedelic in the formulation is delivered to one or more of the group consisting of cerebrospinal fluid, the central nervous system, and a region, tissue, or organ thereof. In some embodiments, at least about 15% of the therapeutic psychedelic in the formulation is delivered to one or more of the group consisting of cerebrospinal fluid, the central nervous system, and a region, tissue, or organ thereof. In some embodiments, at least about 20% of the therapeutic psychedelic in the formulation is delivered to one or more of the group consisting of cerebrospinal fluid, the central nervous system, and a region, tissue, or organ thereof. In some embodiments, at least about 25% of the therapeutic psychedelic in the formulation is delivered to one or more of the group consisting of cerebrospinal fluid, the central nervous system, and a region, tissue, or organ thereof. In some embodiments, at least about 30% of the therapeutic psychedelic in the formulation is delivered to one or more of the group consisting of cerebrospinal fluid, the central nervous system, and a region, tissue, or organ thereof. In some embodiments, at least about 35% of the therapeutic psychedelic in the formulation is delivered to one or more of the group consisting of cerebrospinal fluid, the central nervous system, and a region, tissue, or organ thereof. In some embodiments, at least about 40% of the therapeutic psychedelic in the formulation is delivered to one or more of the group consisting of cerebrospinal fluid, the central nervous system, and a region, tissue, or organ thereof. In some embodiments, at least about 45% of the therapeutic psychedelic in the formulation is delivered to one or more of the group consisting of cerebrospinal fluid, the central nervous system, and a region, tissue, or organ thereof. In some embodiments, at least about 50% of the therapeutic psychedelic in the formulation is delivered to one or more of the group consisting of cerebrospinal fluid, the central nervous system, and a region, tissue, or organ thereof. In some embodiments, at least about 55% of the therapeutic psychedelic in the formulation is delivered to one or more of the group consisting of cerebrospinal fluid, the central nervous system, and a region, tissue, or organ thereof. In some embodiments, at least about 60% of the therapeutic psychedelic in the formulation is delivered to one or more of the group consisting of cerebrospinal fluid, the central nervous system, and a region, tissue, or organ thereof. In some embodiments, at least about 65% of the therapeutic psychedelic in the formulation is delivered to one or more of the group consisting of cerebrospinal fluid, the central nervous system, and a region, tissue, or organ thereof. In some embodiments, at least about 70% of the therapeutic psychedelic in the formulation is delivered to one or more of the group consisting of cerebrospinal fluid, the central nervous system, and a region, tissue, or organ thereof. In some embodiments, at least about 75% of the therapeutic psychedelic in the formulation is delivered to one or more of the group consisting of cerebrospinal fluid, the central nervous system, and a region, tissue, or organ thereof. In some embodiments, at least about 80% of the therapeutic psychedelic in the formulation is delivered to one or more of the group consisting of cerebrospinal fluid, the central nervous system, and a region, tissue, or organ thereof. In some embodiments, at least about 85% of the therapeutic psychedelic in the formulation is delivered to one or more of the group consisting of cerebrospinal fluid, the central nervous system, and a region, tissue, or organ thereof. In some embodiments, at least about 90% of the therapeutic psychedelic in the formulation is delivered to one or more of the group consisting of cerebrospinal fluid, the central nervous system, and a region, tissue, or organ thereof.

The method disclosed herein can provide delivery of a greater amount of the therapeutic psychedelic to one or more of the group consisting of cerebrospinal fluid, the central nervous system, and a region, tissue, or organ thereof compared to intravenous delivery of an identical dose of the therapeutic psychedelic.

The therapeutic psychedelic can provide a therapeutic effect upon delivery to the cerebrospinal fluid, the central nervous system, or region, tissue, or organ thereof via the method disclosed herein.

In some embodiments, the subject has been diagnosed with a disease or disorder related to the central nervous system.

In some embodiments, the delivery of the therapeutic psychedelic provides effective treatment for Alzheimer's disease, Parkinson's disease, a cancer of the brain, a cancer of the CNS, a non-CNS disorder, an immunological disorder, an oncological disorder, a hematological disorder, an opthalmological disorder, a neurological disorder, a respiratory disorder, a cardiovascular disorder, an infectious disease, vitamin deficiency, multiple sclerosis, improvement of uterine contractions, postmenopausal osteoporosis, cryptorchism, endometriosis, central precocious puberty (CPP), Primary nocturnal enuresis, central cranial diabetes insipidus, nocturia, cranial diabetes insipidus or nocturia associated with multiple sclerosis, stroke, epilepsy, central nervous system trauma, a metabolic disorder, an infectious agent (i.e. Naegleria fowleri) or protein (i.e. Creutzfeldt Jacobs disease), a malignant tumor, a benign tumor, glioblastoma, anosmia, or an endocrine disorder.

In some embodiments, the delivery of the therapeutic psychedelic provides effective treatment for depression, treatment-resistant depression (TRD), post-traumatic stress disorder (PTSD), alcohol use disorder (AUD), generalized anxiety disorder (GAD), bipolar disorder, bipolar disorder II (BDII), postpartum depression (PPD), fragile X syndrome, opioid withdrawal, anorexia nervosa, bulimia nervosa, binge eating disorder, major depressive disorder (MDD), fibromyalgia, acute suicidal ideation and behavior (ASIB), cluster headaches, obsessive compulsive disorder, adjustment disorder, attention deficit hyperactivity disorder (ADHD), ischemic stroke, or autism spectrum disorder (ASD).

The method disclosed herein can minimize shear forces exposed to the therapeutic psychedelic.

In some embodiments, the formulation is delivered without exposing the therapeutic psychedelic to shear forces sufficient to damage a significant portion of the therapeutic psychedelic, without aerosolizing the formulation, or by passing the formulation through a shear disintegrating tip.

In some embodiments, the therapeutic psychedelic bypasses the blood-brain barrier, the blood-cerebrospinal fluid barrier, or the arachnoid membrane.

In some embodiments, the subject is a mammal. In some embodiments, the subject is a non-human primate. In some embodiments, the subject is a human.

### II. DEVICE

In another aspect, provided herein is a device for olfactory delivery of a therapeutic psychedelic to an olfactory region of a subject, the device comprising: (a) a housing comprising an insertable portion comprising a distal end, and a proximal end; and (b) a subject-engaging portion which engages a columella region of the subject to seat the distal end of the insertable portion within an ejection zone of a nasal channel of the subject; wherein the device is configured to deliver a formulation comprising a therapeutically effective amount of the psychedelic to the olfactory region of the subject. In another aspect, provided herein is a device for olfactory delivery of a therapeutic psychedelic to an olfactory region of a subject. The device can comprise a housing comprising an insertable portion comprising a distal end and a proximal end. The device can comprise a subject-engaging portion which engages a columella region of the subject. The subject-engaging portion can seat the distal end of the insertable portion within an ejection zone of a nasal channel of the subject. The device can be configured to deliver a formulation comprising a therapeutically effective amount of the psychedelic to the olfactory region of the subject.

In some embodiments, the device dispenses the formulation as a laminar jet.

In some embodiments, application of pressure by the subject-engaging portion to the columella region of the subject enables and/or causes delivery of the formulation to the subject from the insertable portion.

In some embodiments, the insertable portion comprises a dispensing element for delivery of the formulation to the olfactory region of the subject.

The housing can comprise two insertable portions. In some embodiments, the two insertable portions comprise at least one dispensing element, each insertable portion for insertion into a nasal channel of the subject, wherein, upon insertion of at least one insertable portion into a nasal channel of the subject, the at least one insertable portion engages tissue within the nasal channel to open or expand an internal nasal valve of the subject thereby positioning the at least one dispensing element for delivery of the formulation to the subject. In some embodiments, the subject engaging portion comprises a trigger, wherein upon application of pressure to the subject engaging portion, the trigger permits actuation of the device to deliver the formulation to the subject through the dispensing element. The device can be used with the methods disclosed herein.

### III. DEFINITIONS

Unless defined otherwise, all technical and scientific terms used herein have the same meaning as commonly understood by one of ordinary skill in the art to which this disclosure pertains.

The term “about” or “approximately” when used in reference to a particular recited value, means the value may vary from the recited value by no more than 10%, 5%, 2% or 1%. Where a particular value is recited, it can be understood that the value is modified by the term “about” or “approximately”, unless indicated otherwise.

The term “comprise”, “comprising”, or the like means additional elements or components other than those recited may be present. Other terms such as “include”, “contain”, “have” and the like have similar meaning.

The term “consist of”, “consisting of” or the like means no additional component is present.

As used herein, the singular forms “a,” “an,” and “the” include plural references, unless indicated otherwise. For example, a reference to “a molecule” can be a reference to more than one molecule.

The term “or” is used to mean “and/or”, unless it is indicated explicitly to refer to alternatives only. It should also be noted that the term “or” is generally employed in its sense including “and/or” unless the content clearly dictates otherwise. These terms can convey that any combination is specifically contemplated. Solely for illustrative purposes, the expression “A, B, and/or C” can mean A individually; B individually; C individually; A and B; B and C; A and C; and A, B, and C.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of a disease or disorder.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

### Example 1—Laminar Fluid Ejection Method

The primary goal of the study was to develop a method of ejecting fluid through a cannula for deposition at the olfactory cleft. In general, all devices consisted of a 1 mL polycarbonate syringe filled to 0.20 mL of formulation. The syringe was capped with a cannula (inner diameter=1.35 mm) and mounted on a carbon-fiber reinforced mechanism. A coil spring housed within the mechanism provided the force to compress the syringe. The spring preload was adjustable with the use of an external threaded collar and shims. The spring surrounded an orifice-based damper that could be filled with different weight silicone oils and orifice plates with different sized holes and quantity of holes. The mechanism used a simple trigger that released a syringe plunger guided by aluminum rails. This bar pushed the plunger shaft of the syringe. The syringe was held accurately and firmly in place in front of the mechanism.

Aperture testing was conducted to evaluate the coherence of fluid post-ejection and to visually assess the laminar qualities of the ejected fluid. Fluids at two different viscosities (1 cP and 50 cP) were tested, each at a range of ejection velocities (2 m/s-27.7 m/s). Device settings for a selection of velocities within this range were determined experimentally prior to conducting aperture testing. Velocity was determined using high-speed camera-footage of ejected fluid traveling a fixed distance from the tip of the canula. A small aperture (5.64 mm in diameter) was placed 25 mm away from the tip of the canula. The canula was supported by magnetic supports to reduce wobbling. A catching tray was placed on the opposite side of the aperture. The percent mass transferred from the syringe to the catching tray was calculated for each test-run (Mass in catching tray/Mass Ejected from syringe*100). Tests of each combination of viscosity and speed were repeated three times. The mean percent mass transferred for each condition is presented in Table 1. (Note that the ejected mass is smaller than the total fill mass, as it does not include the residual left in the device. The residual left in the device did not meaningfully differ across conditions: 13.6% for 1 cP, 10.4% for 50 cP).

TABLE 1
Aperture Testing

| Viscosity | Velocity | % Mass |
|---|---|---|
| (cP) | (m/s) | Transfer |
| 1.0 | 3.7 | 92.8% |
| 50.0 | 3.1 | 96.0% |
| 1.0 | 27.7 | 78.1% |
| 50.0 | 27.7 | 98% |

Percent mass transferred from the device through a small 5.64 mm aperture and into a catching tray for low/high fluid viscosities and velocities.

In general, higher speeds were associated with reduced mass transfer, possibly due to increased cannula movement and splatter off the aperture edge. Qualitatively, it was observed that the device ejected fluid in a cohesive stream with a narrow diameter, especially compared to the plume emitted from a traditional spray device.

Next, a series of in-vitro deposition tests was conducted to determine the range of velocities that would optimally deposit fluid in the olfactory cleft of the human nasal passages. To test this, the mechanism was inserted into a transparent 3D-printed model of the nasal cavity. Optimal delivery was considered to be in the target zone, which represents the approximate location of the olfactory cleft in the model. If ejected too slowly, the fluid would deposit anterior to the front boundary of the target zone; too quickly and it would deposit beyond the back boundary. Two fluid viscosities (1 cP and 50 cP) were tested at each of three different insertion angles (30, 35, 40 degrees from the vertical). The cannula's insertion depth was held constant at 37.5 mm. The minimum velocity necessary to reach the Front Boundary (vf), the olfactory cleft (vopt), and the back boundary (vb), for each of the viscosities and deposition angles are presented in Table 2.

TABLE 2
In-vitro Deposition Testing

| Angle | Viscosity | Vf | Vopt | Vb |
|---|---|---|---|---|
| (°) | (cP) | (m/s) | (m/s) | (m/s) |
| 30 | 1 | 1.1 | 1.6 | 1.9 |
| 35 | 1 | 1.0 | 1.1-1.9 | 2.2 |
| 40 | 1 | 1.0 | 1.1-1.9 | 2.8 |
| 30 | 50 | 4.7 | 13.9-15.8 | 18.6 |
| 35 | 50 | 4.7 | 11.0-15.8 | 18.6 |
| 40 | 50 | 4.7 | 11.0-15.8 | 18.6 |

Table presents optimal velocities (Vopt) for fluids of low/high viscosity across three device angles. The Vf and Vb columns show the velocity limits at which the deposition was in front of or behind the target area, respectively.

For velocities within the optimal range, residence times were observed to exceed 10 minutes. Notably, angles of 35-40 degrees permitted a wider range of velocities, but using an angle of 30-degrees did not dramatically shift the optimal velocity. This suggested the device is adaptable to a wide range of angles. Optimal velocities (+/−20%) were found to be 1.6 m/s for 1 cP fluid and 14.0 m/s for 50 cP fluid. These optimal velocities may be associated with laminar flow.

A Reynold's number (Rₑ=p*V*L/μ, where ρ is the density of the fluid, V is the velocity of the flow, L is the diameter of the passage, and μ is the dynamic viscosity of the fluid) can be computed to quantify whether a fluid exhibits laminar or turbulent flow. The Reynold's number for water at a velocity of 1.6 m/s and viscosity of 1 cP is approximately 2,160 with a 1.35 mm diameter (density of water is approximately 1000 kg/m³). Similarly for a fluid of 50 cP (with the same density as water), the Reynold's number at 15 m/s is approximately equal to 405 with a 1.35 mm diameter. Both Reynold's numbers fall below 2,300 which is the commonly accepted threshold for laminar flow.

### Example 2 In-Vivo Evaluation of Laminar Fluid Ejection Method

The primary goal of these studies is to visualize delivery to the olfactory cleft (first with technetium-99, and then with methylene blue). The device was tuned to eject the fluid at a specific velocity. This velocity was chosen based on the viscosity of the fluid and the studies in Example 1, to maximize the extent of laminar flow.

Visualizing Olfactory Cleft Delivery with Technetium-99

Participants: Nine healthy participants (ages 19+) with no history of abnormal nasal or sinus symptoms or contraindications for nasal cannulation, Magnetic Resonance Imaging (MRI), or Single-Photon Emission Computed Tomography (SPECT). Participants completed one study visit undergoing magnetic resonance imaging (MRI) and single-photon emission computed tomography (SPECT) to visualize the deposition of a radiolabeled tracer (technetium-99) in their nasal passages. In total, nine participants were enrolled in the study, and eight completed all study procedures (Participant 1.02 withdrew before completing all imaging procedures). All study procedures were approved by the Horizon Health Network's Human Research Protection Program. Prior to participating in the study, all participants were assessed by a licensed otolaryngologist physician to confirm their eligibility. Study Design: On Day 1 of the study, anatomical (T1-weighted) magnetic resonance images were collected from participants at the Moncton MRI clinic (Moncton, NB, Canada) using a Siemens Skyra VD13 3T scanner. MRI data provided anatomical information regarding soft tissue structure in the nasal cavity and the location of each participant's olfactory region.

On Day 2 of the study, a saline solution including the technetium-99 radiotracer (mean dose 4.6 mCi) was delivered to participants using the Laminar Fluid Ejection method described above. Day 2 study procedures were conducted at the Nuclear Imaging Department of Saint John Regional Hospital (Saint John, NB, Canada). At the time of delivery and for five minutes afterwards, 2D SPECT Flow images were acquired every three seconds using a gamma camera. Flow images were acquired in a single sagittal plane (128×128 with voxels 4.8 mm diameter).

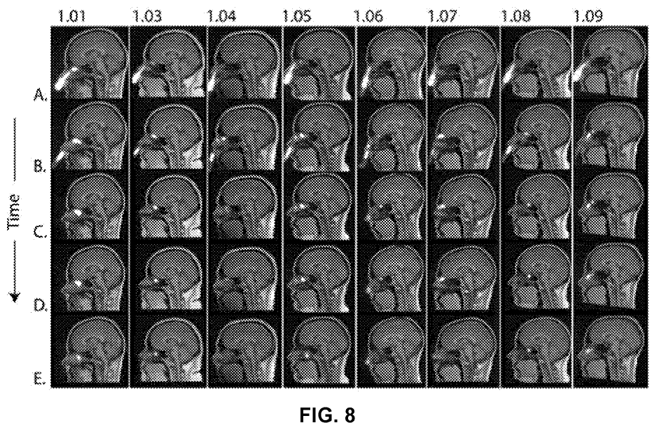

Image Analysis: Image analysis was completed in Vivoquant (4.0). SPECT Flow data was resampled to match MRI resolution (320×320 with voxels 1.918 mm isotropic) and manually registered to T1 images in x and y space with minimal rotation in the z-plane. This procedure enabled landmarking to nasal passage characteristics minimally visible in the SPECT data alone. Flow data was visually inspected to determine (1) the point in the time series when the bolus was dispensed, (2) the time of cannula removal, (3) the median time point in the series where the bolus initially lodged between points 2 and 4, (4) the time point at which the bolus began additional migration, and (5) the time point of the final image acquired during the five-minute SPECT series. All subjects' SPECT Flow images are presented in FIG. 8.

Results: In seven out of eight participants imaging results demonstrated delivery of the radiotracer to the cribriform area. The radiotracer was detected in the cribriform area without any movement for at least 1.5 minutes in six of those seven participants. Additionally, clearance of the radiotracer was minor for the duration of the study for six out of those seven cases. In the two cases where sub-optimal outcomes were reached, it was the result of (i) bolus delivery anterior and superior to the cribriform (Participant 1.07), and (ii) bolus that cleared quickly after reaching the target, possibly due to the bolus remaining well intact as it directly impacted the cribriform (Participant 1.05). See Table 3 for a summary of the results in each participant.

TABLE 3
Visualizing Olfactory Cleft Delivery with Technetium-99

| | Cribriform | |
|---|---|---|
| ID | Reached? | Notes |
| 1.01 | Yes | |
| 1.03 | Yes | |
| 1.04 | Yes | Some bolus deflected off top of nasal |
| | | bone into lower turbinate |
| 1.05 | Yes | Extremely fast cleance, possibly due |
| | | to the acute angle of administration. |
| 1.06 | Yes | |
| 1.07 | No | Bolus arrived superior/anterior to |
| | | cribriform and most returned down |
| | | the nasal passage. |
| 1.08 | Yes | |
| 1.09 | Yes | |

Visualizing Olfactory Cleft Delivery with Methylene Blue

Participants: Five healthy participants (ages 19+) with no history of sinonasal symptoms and no evidence of nasal inflammation were recruited for this study. Prior to recruitment of participants study procedures were approved by Providence Health Care research ethics board, (British Columbia, Canada) and conducted under Health Canada Investigational Testing Authorization #314993. All participants were assessed by a licensed otolaryngologist physician to confirm their eligibility. Participants completed two study visits during which a licensed otolaryngologist physician administered 0.1 mL of the visual dye tracer, methylene blue.

Study Design: During the first visit, the methylene blue was administered using the Laminar Fluid Ejection method (1.5 mm cannula; velocity=4.5 m/s). During the second visit, methylene blue was administered using a conventional nasal spray device (Pharma systems Item #10272, UPC: 063636802714). The Pharma systems spray device was used in these experiments because it was the device normally used by the compounding pharmacy for intranasal delivery. It was the device patients and clinicians were used to at this clinic.

Following delivery, the physician used a nasal endoscope to image the deposition of methylene blue to the olfactory cleft at up to five time points (1, 5, 8, 12, and 15 minutes following delivery). From each image, the physician judged whether at least 50% of the methylene blue fluid was delivered to the olfactory cleft. If <50% of the methylene blue was judged to have deposited in the olfactory cleft, imaging was stopped and that session's trial was completed.

Results: Four of the five participants demonstrated successful delivery of >50% of the methylene blue to the olfactory cleft using the Laminar Fluid Ejection method. In each of the five participants, the conventional nasal spray failed to deliver at least 50% of the dye to the olfactory cleft (Table 4).

TABLE 4
Visualization of Delivery to the Olfactory
Clef Using Methylene Blue

| | |
|---|---|
| LFE Method | Nasal Spray |

| | | Olfactory | | Olfactory |
|---|---|---|---|---|
| | Attempts | Residence | Attempts | Residence |
| ID | Made | Time (min.) | Made | Time (min.) |
| 2.01 | 1 | 15 | 1 | 0 |
| 2.02 | 2 | 8 | 1 | 0 |
| 2.03 | 1 | 5 | 1 | 0 |
| 2.04 | 5 | 0 | 1 | 0 |
| 2.05 | 1 | 1 | 1 | 0 |

Results from in-human tests of the Laminar Fluid Ejection (LFE) method using the dye methylene blue.

### Example 3: Effect of Administration Routes on Olfactory Bulb Delivery in Rats

Background: The ability of a drug to reach its target can be affected by its route of administration. Standard routes of administration include intravenous (IV) and intranasal (IN). Intravenous injections can cause patient distress and require a trained phlebotomist to administer the drug. Intranasal sprays are often preferred by patients because of their ease of use, but suffer from imprecise dosing and delivery. The goal of this example was to compare nasal cannula delivery (OD) with the traditional IV and IN administration routes.

Procedure: The three routes of administration were tested on rats. A compound formulation of ketamine (5.0 mg/kg), naloxone (0.5 mg/kg), diazepam (2.0 mg/kg), and ondansetron (1.0 mg/kg) was administer. 15 minutes after the drug was administered, rats were sacrificed and samples were taken from plasma, the olfactory bulb, and the brain. To assess each route of administration's efficacy, drug concentrations in each tissue were quantified using liquid chromatography-mass spectrometry (LCMS).

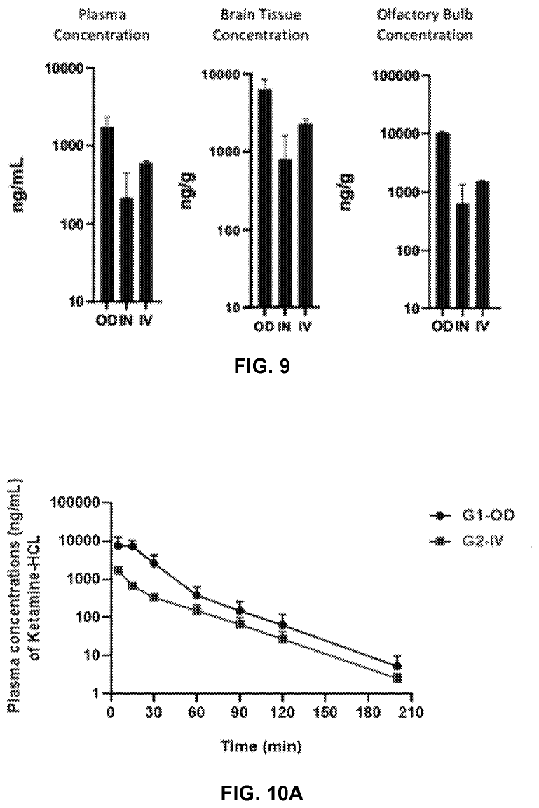

Results (See FIG. 9): Preliminary findings from this pilot study suggest that the novel OD route of administration delivers drugs to the plasma, olfactory bulb, and brain more effectively than the traditional IN route of administration. When comparing the OD route of administration to IV, the two methods perform similarly in delivering drug to plasma, but the OD route may out-perform IV in delivering drug to the olfactory bulb and brain.

FIG. 9 shows the concentration of four compounds in plasma (left) brain tissue (center) and the olfactory bulb (right) 15 minutes after administration via one of three methods: olfactory delivery (OD) via cannula, traditional intranasal delivery (IN), and traditional intravenous delivery (IV).

This example suggests that the OD route of administration can perform at least on-par with the IV route of administration, and can outperform the IN route of administration. For OD administration, concentrations were highest in the olfactory bulb. Olfactory bulb concentrations were almost an order of magnitude larger than brain tissue concentrations, which in turn were almost an order of magnitude larger than plasma concentrations. It is intriguing that the OD and IV routes of administration show lower standard deviations than the IN route. This suggests that even in a laboratory setting when trained scientists are administering compounds, the IN route still suffers from some inconsistent dosing and delivery.

This example demonstrates that targeted intranasal administration of therapeutic psychedelics (such as ketamine) to the olfactory cleft offers improved delivery to brain tissue compared to traditional intranasal sprays.

### Example 4: Plasma and Tissue Concentrations of Ketamine Via Olfactory Delivery Compared to Intravenous Administration in Rodents

Drug Administration and Sample Collection: Intravenous (IV) administration was conducted by 100 μL tail vein injection. Olfactory delivery (OD) administration was conducted by a. 0.699 mm diameter cannula inserted into the right nostril with a minimal angle of 20-degrees to the target of the correct meatus.

Sample Preparation: An aliquot of 30 μL plasma was spiked into a 1.5 mL tube, and 120 μL of acetonitrile containing internal standard were added for protein precipitation. The mixture was vortexed, centrifuged at 14000 rpm for 10 min. 50 μL of supernatant was resolved with 150 μL H₂O, then injected for LC-MS/MS analysis. Brain or olfactory sample was homogenized with ice-cold phosphate buffer saline (pH 7.4) at a ratio of 4 (buffer): 1 (tissue) (v/w). An aliquot of 30 μL homogenate was spiked into 1.5 mL tube, and 120 μL of acetonitrile containing internal standard was added for protein precipitation. The mixture was vortexed, centrifuged at 14000 rpm for 10 min. 50 μL of supernatant was mixed with 150 μL of water and the final solution was injected for LC-MS/MS analysis.

Liquid Chromatography/Mass Spectrometry: An Ultra Performance Liquid Chromatography (UPLC) chromatographic system (Waters), equipped with an AB Sciex QTRAP 6500 mass spectrometer was used to analyze the samples. Chromatographic separation was achieved on the Waters HSS T3 column 50*2.1 mm ID, 1.8 μm, and a gradient mobile phase changing from 90% H₂O (0.1% formic acid) (A) and 90% acetonitrile (0.1% formic acid) (B). The flow rate was maintained at 0.7 mL/min. Analyst 1.6 software packages (Applied Biosystems) were used to control the LC/MS/MS system, as well as for data acquisition and processing. A calibration curve was constructed in blank olfactory bulb homogenate.

Time course experiment and tissue concentration: 12 male Sprague Dawley rats (Age M+/−SD, Weight: 176.27-188.03 g) were administered Ketamine-HCl via IV or OD administration (resulting in two experimental groups, each with n=6). Ketamine-HCl was formulated according to the following procedures: For Ketamine-HCL 5 mg/kg OD: 24.64 mg Ketamine-HCL was added to 0.426 ml of saline, mixed well, and dissolved, with a solution concentration of 50 mg/ml. For Ketamine-HCL 5 mg/kg IV: 0.20 ml of 50 mg/ml Ketamine-HCL solution, was added to 0.60 ml of saline and mixed well to achieve a solution concentration of 12.5 mg/ml. Plasma time courses for 200 minutes were generated. Concentration of compound in brain, olfactory bulb, and plasma at t=200 minutes was quantified using LC/MS/MS.

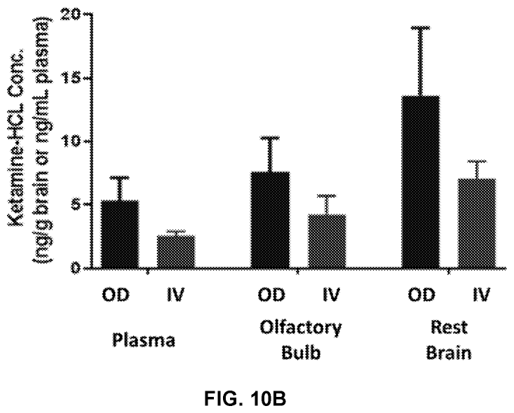

Results are shown in FIGS. 10A-10B and Table 5. FIG. 10A shows that a higher plasma concentration of ketamine can be achieved with OD than IV.

TABLE 5
Mean plasma concentration at different time points

Plasma

| | OD | IV |
|---|---|---|
| Time (min) | ng/ml | ng/ml |
| 5 | 7597 | 1713 |
| 15 | 7253 | 686 |
| 30 | 2630 | 330 |
| 60 | 383 | 150 |
| 90 | 149 | 65.8 |
| 120 | 63 | 27.1 |
| 200 | 5.3 | 2.53 |

### Example 5: Additional Examples of Administration of Therapeutic Psychedelics Via Olfactory Delivery

Studies similar to Example 4 will be performed to demonstrate olfactory delivery of one or more psychedelics selected from the group comprising R-ketamine, S-ketamine, psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, a serotonin antagonist, or an analog or derivative of any of the preceding.

Non-limiting examples of cannabinoids include cannabidiol, tetrahydrocannabinol, cannabinol, cannabigerol, cannabichromene, tetrahydrocannabivarin, cannabidivarin, and analogs and derivatives thereof such as acids and esters.

Non-limiting examples of 5-HT2C receptor agonists include lorcaserin, vabicaserin, and analogs and derivatives thereof.

Non-limiting examples of dopamine antagonists include Chlorpromazine, Fluphenazine, Haloperidol, Loxapine, Molindone, Perphenazine, Pimozide, Prochlorperazine, Thiothixene, Thoridazine, Trifluoperazine, Aripiprazole, Asenapine, Brexpiprazole, Cariprazine, Clozapine, Iloperidone, Lumateperone, Lurasidone, Olanzapine, Quetiapine, Paliperidone, Pimavanserin, Risperidone, Ziprasidone, Amisulpride, Metoclopramide, Droperidol, Haloperidol, Chlorpromazine, Prochlorperazine, Olanzapine, and analogs and derivatives thereof.

Non-limiting examples of serotonin antagonist include etoperidone, lorpiprazole, lubazodone, mepiprazole, nefazodone, and trazodone and analogs and derivatives thereof.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

The scope of the claims should not be limited by the embodiments and examples, but should be given the broadest interpretation consistent with the description as a whole.

What is claimed is:

1. A method of delivering a therapeutic psychedelic to a subject in need thereof, the method comprising:
a. selectively delivering a formulation comprising a therapeutically effective amount of the therapeutic psychedelic to an olfactory region of the subject;
wherein at least a portion of formulation is delivered to one or more of the group consisting of cerebrospinal fluid, the central nervous system or a region, tissue, or organ thereof, and a non-CNS target.

2. The method of claim 1, wherein the formulation is delivered as a liquid jet.

3. The method of any one of claims 1-2, wherein the formulation is delivered as a laminar flow.

4. The method of any one of claims 1-3, wherein the formulation is delivered as a flow having a Reynold's number of 2300 or less.

5. The method of any one of claims 1-4, wherein the formulation is ejected from a dispensing element, wherein at least about 70%, at least about 75%, at least about 80%, at least about 85%, at least about 90%, or at least about 95% of the formulation passes through a circular opening having a diameter of between about 5 mm and about 6 mm at a distance of about 25 mm from where the formulation is ejected from the dispensing element.

6. The method of any one of claims 1-5, wherein the formulation has a viscosity of between about 0.5 cP and about 100 cP.

7. The method of any one of claims 1-5, wherein the formulation has a viscosity of between about 0.5 cP and about 50 cP.

8. The method of any one of claims 1-5, wherein the formulation has a viscosity of between about 0.5 cP and about 10 cP.

9. The method of any one of claims 1-5, wherein the formulation has a viscosity of between about 45 cP and about 55 cP.

10. The method of any one of claims 1-9, wherein the formulation is ejected from the dispensing element at a velocity of between about 1 m/s and about 30 m/s.

11. The method of any one of claims 1-9, wherein the formulation is ejected from the dispensing element at a velocity of between about 2 m/s and about 4 m/s.

12. The method of any one of claims 1-9, wherein the formulation is ejected from the dispensing element at a velocity of between about 25 m/s and about 30 m/s.

13. The method of any one of claims 1-4, wherein the formulation is ejected from a dispensing element, wherein at least 75% of the formulation passes through a circular opening having a diameter of between about 5 mm and about 6 mm at a distance of about 25 mm from where the formulation is ejected from the dispensing element, wherein the formulation has a viscosity of between about 0.5 cP and about 50 cP, and wherein the formulation is ejected from the dispensing element at a velocity of between about 1 m/s and about 30 m/s.

14. The method of any one of claims 1-13, wherein at least about 50%, at least about 60%, at least about 70%, at least about 80%, or at least about 90% of the formulation is delivered to the olfactory region of the subject.

15. The method of any one of claims 1-14, wherein delivery of the therapeutic psychedelic to the olfactory region of the subject results in a plasma concentration of the therapeutic psychedelic about 30 min after delivery that is at least about 2 times, at least about 4 times, at least about 6 times, at least about 8 times, or at least about 10 times of a plasma concentration of the therapeutic psychedelic when about the same dose is delivered intravenously.

16. The method of any one of claims 2-4, wherein the delivering the formulation as a laminar flow, a liquid jet, or a flow having a Reynold's number of 2300 or less: a) increases on target delivery of the composition to the target region, b) decreases off target delivery of the composition to the nasal cavity, or c) both, compared to delivering the formulation with a spray ejection profile.

17. The method of any one of claims 1-16, wherein selectively delivering the formulation to the olfactory region of the subject comprises ejecting the formulation from an ejection zone in a nasal cavity of the subject, wherein the ejection zone is:
(a) 0 mm to 30 mm superior to a horizontal line that intersects the anterior aspect of the internal nasal valve, and
(b) 0 mm to 20 mm anterior to an inclined line that intersects the anterior aspect of the middle turbinate and the posterior aspect of the vestibule.

18. The method of claim 17, wherein the ejection zone is further:
i. 0 mm to 40 mm inferior to a horizontal line that is parallel to the inferior aspect of the olfactory cleft,
ii. 0 mm to 20 mm posterior to the internal nasal dorsum,
iii. 10 mm to 50 mm superior to a horizontal line that intersects the inferior aspect of the columella,
iv. 0 mm to 30 mm superior to a horizontal line that intersects the superior aspect of the inferior turbinate,
v. 0 mm to 3 mm from the septum, or
vi. any combination thereof.

19. The method of claim 17 or 18, wherein delivering the formulation from the ejection zone: a) increases on target delivery of the composition to the target region, b) decreases off target delivery of the composition to the nasal cavity, or c) both, compared to dispensing the formulation outside the ejection zone.

20. The method of any one of claims 17-19, wherein the formulation is delivered by a device comprising the dispensing element, wherein the device is inserted into a nasal cavity of the subject at an angle of between about 30 degrees and about 40 degrees from a vertical line, wherein the vertical line is perpendicular to a horizontal line that is parallel to an inferior aspect of the olfactory cleft of the subject.

21. The method of any one of claims 1-20, wherein the formulation delivered to the olfactory region remains at the olfactory region for at least about 10 seconds, at least about 30 seconds, at least about 1 min, at least about 2 min, at least about 3 min, at least about 4 min, at least about 5 min, at least about 6 min, at least about 7 min, at least about 8 min, at least about 9 min, at least about 10 min, at least about 11 min, at least about 12 min, at least about 13 min, at least about 14 min, or at least about 15 min.

22. The method of any one of claims 1-21, wherein the therapeutic psychedelic is ketamine, R-ketamine, S-ketamine, psilocin or psilocybin, a cannabinoid, 3,4-Methylenedioxy methamphetamine (MDMA), lysergic acid diethylamide (LSD), N,N-Dimethyltryptamine (DMT), ibogaine, isoprocin glutarate, a 5-HT2C receptor agonist, a dopamine antagonist, or a serotonin antagonist.

23. The method of any one of claims 1-21, wherein the therapeutic psychedelic is an analog of one of the group consisting of ketamine, R-ketamine, S-ketamine, psilocin or psilocybin, a cannabinoid, 3,4-Methylenedioxymethamphetamine (MDMA), lysergic acid diethylamide (LSD), N,N-Dimethyltryptamine (DMT), ibogaine, isoprocin glutarate, a 5-HT2C receptor agonist, a dopamine antagonist, and a serotonin antagonist.

24. The method of claim 22 or 23, wherein the cannabinoid is selected from the group consisting of: cannabidiol, tetrahydrocannabinol, cannabinol, cannabigerol, cannabichromene, tetrahydrocannabivarin, cannabidivarin, and analogs and derivatives thereof.

25. The method of claim 22 or 23, wherein the 5-HT2C receptor agonist is selected from the group consisting of: lorcaserin, vabicaserin, and analogs and derivatives thereof.

26. The method of claim 22 or 23, wherein the dopamine antagonist is selected from the group consisting of: Chlorpromazine, Fluphenazine, Haloperidol, Loxapine, Molindone, Perphenazine, Pimozide, Prochlorperazine, Thiothixene, Thoridazine, Trifluoperazine, Aripiprazole, Asenapine, Brexpiprazole, Cariprazine, Clozapine, Iloperidone, Lumateperone, Lurasidone, Olanzapine, Quetiapine, Paliperidone, Pimavanserin, Risperidone, Ziprasidone, Amisulpride, Metoclopramide, Droperidol, Haloperidol, Chlorpromazine, Prochlorperazine, Olanzapine, and analogs and derivatives thereof.

27. The method of any one of claims 1-26, wherein the therapeutic psychedelic comprises ketamine, or a derivative or analog thereof.

28. The method of any one of claims 1-27, wherein the therapeutic psychedelic is conjugated to a therapeutic agent.

29. The method of claim 28, wherein the therapeutic agent is a drug or a radioisotope.

30. The method of any one of claims 1-29, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the therapeutic psychedelic in the formulation is delivered to one or more of the group consisting of cerebrospinal fluid, the central nervous system, or a region, tissue, or organ thereof.

31. The method of any one of claims 1-30, wherein the method provides delivery of a greater amount of the therapeutic psychedelic to one or more of the group consisting of cerebrospinal fluid, the central nervous system, or a region, tissue, or organ thereof than provided by intravenous delivery of an identical dose of the therapeutic psychedelic.

32. The method of any one of claims 1-31, wherein the therapeutic psychedelic provides a therapeutic effect upon delivery to the cerebrospinal fluid, the central nervous system, or region, tissue, or organ thereof.

33. The method of any one of claims 1-32, wherein the subject has been diagnosed with a disease or disorder related to the central nervous system.

34. The method of any one of claims 1-33, wherein delivery of the therapeutic psychedelic provides effective treatment for Alzheimer's disease, Parkinson's disease, a cancer of the brain, a cancer of the CNS, a non-CNS disorder, an immunological disorder, an oncological disorder, a hematological disorder, an opthalmological disorder, a neurological disorder, a respiratory disorder, a cardiovascular disorder, an infectious disease, multiple sclerosis, stroke, epilepsy, central nervous system trauma, a metabolic disorder, an infectious agent (i.e. Naegleria fowleri) or protein (i.e. Creutzfeldt Jacobs disease), a malignant tumor, a benign tumor, glioblastoma, anosmia, or an endocrine disorder.

35. The method of any one of claims 1-34, wherein delivery of the therapeutic psychedelic provides effective treatment for depression, treatment-resistant depression (TRD), post-traumatic stress disorder (PTSD), alcohol use disorder (AUD), generalized anxiety disorder (GAD), bipolar disorder, bipolar disorder II (BDII), postpartum depression (PPD), fragile X syndrome, opioid withdrawal, anorexia nervosa, bulimia nervosa, binge eating disorder, major depressive disorder (MDD), fibromyalgia, acute suicidal ideation and behavior (ASIB), cluster headaches, obsessive compulsive disorder, adjustment disorder, attention deficit hyperactivity disorder (ADHD), ischemic stroke, or autism spectrum disorder (ASD).

36. The method of any one of claims 1-35, wherein the formulation is delivered without exposing the therapeutic psychedelic to shear forces sufficient to damage a significant portion of the therapeutic psychedelic, without aerosolizing the formulation, or by passing the formulation through a shear disintegrating tip.

37. The method of any one of claims 1-36, wherein the therapeutic psychedelic bypasses the blood-brain barrier, the blood-cerebrospinal fluid barrier, or the arachnoid membrane.

38. The method of any one of claims 1-37, wherein the formulation is delivered via a device comprising:
a. a housing defining first and second insertable portions, each for insertion into a nasal channel of the subject, wherein, upon insertion of the first insertable portion into the nasal channel of the subject, the at least one insertable portion engages tissue within the nasal channel to open or expand an internal nasal valve of the subject thereby positioning at least one of the insertable portions for delivery of the formulation to the olfactory region of the subject; and
b. an actuator which delivers the formulation from the either or both of the insertable portions when the device is actuated.

39. The method of any one of claims 1-38, wherein the non-CNS target is blood.

40. A device for olfactory delivery of a therapeutic psychedelic to an olfactory region of a subject, the device comprising:
a. a housing comprising an insertable portion comprising a distal end, and a proximal end; and
b. a subject-engaging portion which engages a columella region of the subject to seat the distal end of the insertable portion within an ejection zone of a nasal channel of the subject;
wherein the device is configured to deliver a formulation comprising a therapeutically effective amount of the psychedelic to the olfactory region of the subject.

41. The device of claim 40, wherein the device dispenses the formulation as a laminar jet.

42. The device of any one of claims 40-41, wherein the device comprises a compliant dispensing tip comprising a compliant and flexible soft nib.

43. The device of any one of claims 40-42, wherein application of pressure by the subject-engaging portion to the columella region of the subject enables and/or causes delivery of the formulation to the subject from the insertable portion.

44. The device of any one of claims 40-43, wherein the insertable portion comprises a dispensing element for delivery of the formulation to the olfactory region of the subject.
